(19) United States
(12) Patent Application Publication
(10) Pub. No.: US 2026/0285898 A1
(43) Pub. Date: Sep. 24, 2026

(54) SAPONIN DERIVATIVES AS ADJUVANTS AND PHOTOAFFINITY PROBES

(71) Applicant: ASOCIACIÓN CENTRO DE INVESTIGACIÓN COOPERATIVE EN BIOCIENCIAS-CIC BIOGUNE, Derio, Vizcaya (ES)

(72) Inventors: Alberto FERNÁNDEZ TEJADA, Derlo, Vizcaya (ES); Abhijit SAHA, Derlo, Vizcaya (ES); Priscila TONON BASCHIROTTO, Derlo, Vizcaya (ES); Juan ANGUITA, Derlo, Vizcaya (ES)

(21) Appl. No.: 19/476,582

(22) PCT Filed: Apr. 19, 2024
(86) PCT No.: PCT/EP2024/060658

§ 371 Date: Oct. 17, 2025

(30) Foreign Application Priority Data
Apr. 20, 2023 (EP) 23382369.9
Nov. 30, 2023 (EP) 23383237.7

(51) Int. Cl. C07J 63/00; A61K 31/7056; A61K 47/55; A61P 35/00; G01N 33/58

(52) U.S. Cl. CPC C07J 63/008; A61K 31/7056; A61K 47/55; A61P 35/00; G01N 33/58

(57) ABSTRACT

The present application relates to compounds based on a triterpene glycoside saponin scaffold, which bear an acyl chain comprising a photoreactive moiety and a terminal biotin, of general formula (I)

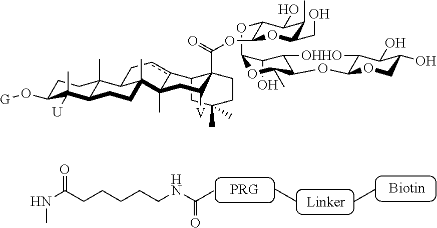

The present application also relates to the use of said compounds and compositions thereof in the treatment of and immunization for diseases such as neurodegenerative and infectious diseases and cancers as well as its use as chemical probe in photoaffinity labeling (PAL) (for photocrosslinking with interacting partners) followed by affinity purification of the covalent complex and MS-based proteomics for target identification and elucidation of the protein receptor.

### TECHNICAL FIELD OF THE INVENTION

The present application is encompassed within the field of chemical immunology. More specifically, it relates to synthetic adjuvants and vaccines based on a triterpene glycoside saponin scaffold and pharmaceutical compositions thereof as well as the use of said compounds and compositions in the treatment of and immunization for diseases such as neurodegenerative and infectious diseases and cancers. The compounds are also useful as chemical probes in photoaffinity labeling (PAL) experiments.

### BACKGROUND OF THE INVENTION

Vaccines have been approved and have improved health care over the last several decades. Attenuated or inactivated pathogens and their toxins have been historically used as vaccines. Modern subunit vaccines based on homogeneous antigens offer more precise targeting and improved safety compared with traditional whole-pathogen vaccines. However, they are also less immunogenic and require an adjuvant to increase the immunogenicity of the antigen and potentiate the immune response (Pifferi, C. et al. Nat. Rev. Chem. 2021, 25 (4), 3-7). Adjuvants enhance antigen-specific immune responses by modulating and enhancing the innate and adaptive (acquired) immunity when delivered together with an antigen (Bergmann-Leitner, E. et al. Vaccines 2014, 2 (2), 252-296). Additionally, they allow the dose of expensive antigens to be decreased, reduce booster immunizations, generate more rapid and durable immune responses, and increase the effectiveness of vaccines in poor responders (Reed, S. G. et al. Nat. Med. 2013, 19 (12), 1597-1608).

However, few adjuvants are of sufficient potency and acceptable toxicity for clinical use. Aluminum-containing adjuvants were the first human vaccine adjuvants approved in clinical use. Aluminum salts, either alone (alum) or in proprietary mixtures (AS04), and oil-in-water emulsions containing squalene (MF59, AS03) have been used as adjuvants in a number of vaccines, but have relatively low potency and significant side effects, respectively. These limitations highlight the urgent need for new, potent and safe adjuvants.

QS-21 is one of the most promising adjuvants currently under investigation. Isolated from Quillaja saponaria tree bark, it is composed of four structural domains: a branched trisaccharide, a quillaic acid triterpene, a bridging linear tetrasaccharide, and a pseudodimeric acyl chain. QS-21 is not a single molecule but a ≈2:1 mixture of two isomers that differ at the terminal sugar of the linear tetrasaccharide domain, the first isomer having a terminal apiose (QS-21-Api) and the second one having a xylose (QS-21-Xyl).

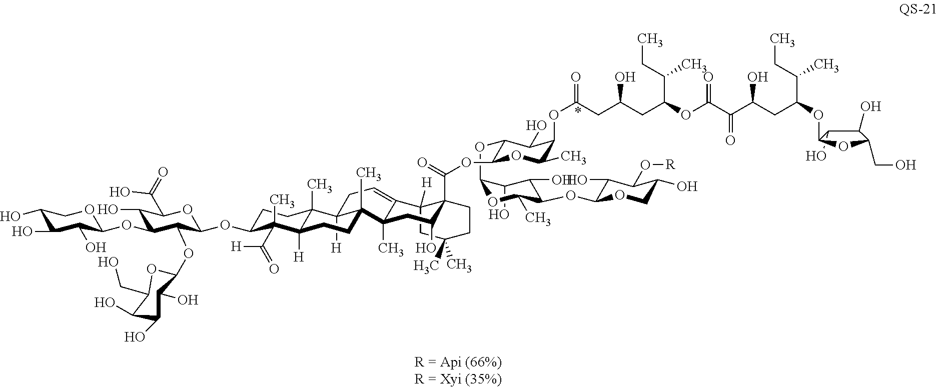

Vaccines that contain QS-21 have been investigated or are under development for several cancers, and for infectious and neurodegenerative diseases (malaria, acquired immunodeficiency syndrome, hepatitis, tuberculosis and Alzheimer's disease). Despite its promise, QS-21 suffers from several liabilities, including limited access from its natural source, toxic side effects and chemical instability through spontaneous hydrolysis of the acyl chain. With the exception of its recent approval as part of the malaria and shingles vaccines, the scarcity, heterogeneity and dose-limiting toxicity of QS-21 have hampered its further use in human vaccines.

Triterpene glycoside saponin-derived adjuvants are disclosed in WO2009/126737, WO2015/184451, WO2017/079582, WO2017/106836, WO2018/191598, WO2018/200645 WO2018/200656 and WO2019/079160.

However, the difficulties in obtaining pure species from Quillaja saponin extract, the difficulties in correctly identifying their structures as well as the poor understanding of the molecular mechanism of action of these saponin-based adjuvants impedes the rational development of analogues with improved efficacy and decreased toxicity. While certain modifications of the QS scaffold have been studied, such as Echinocystic acid or Caullophylogenin variants in which the the native C4-aldehyde substituent of the original quillaic acid core is replaced with a methyl group or an hydroxymethyl group respectively, there is still a need for new derivatives that present potent adjuvant activity and/or low toxicity.

Additionally, it would be also of great interest to develop saponin-based probes for photoaffinity labeling (PAL) studies. PAL is a well-established and valuable technique for studying the interaction of ligands with their target proteins, such as receptor proteins. PAL is useful inter alia to identity unknown targets of ligands, assist in the elucidation of protein structures, functions and conformational changes as well as identify novel or alternative binding sites in proteins Although the technique was initially developed in 1962 by Singh, Thornton, and Westheimer, the basic principles of photoaffinity labeling remain essentially unchanged today. PAL is based on the use of a chemical probe that can covalently bind to its target in response to activation by light. This is made possible by the incorporation of a photoreactive group within an otherwise reversibly binding probe compound. On irradiation with a specific wavelength of light, the photogroup forms a reactive intermediate that rapidly reacts with and binds to the nearest molecule, which ideally will be the target protein.

The general design of photoaffinity probes involves the incorporation of three important functionalities; an affinity/specificity unit, in other words, the small molecule of interest, a photoreactive moiety and an identification/reporter tag. The specificity unit is responsible for reversible binding to target proteins, the photoreactive moiety allows photo-inducible permanent attachment to targets, and the identification component is vital for the detection and isolation of probe-protein adducts. Three types of photoactivating probes are mainly used in photoaffinity labeling: azides, benzophenones and diazirines; whereas biotin is commonly used as identification tag. The probe-protein complex can be isolated via HPLC and analyzed by MS (Yu W et al. Curr Opin Chem Biol. 2022; 69:102173; Kozoriz K et al. Acc. Chem. Res. 2023; 56(1):25-36).

### BRIEF DESCRIPTION OF THE INVENTION

The present invention solves one or more of the aforementioned needs by the provision of new triterpene glycoside saponin adjuvants. The active compounds of the present invention have found to induce significantly higher anti-OVA antibody titers and/or reduced toxicity than the natural QS-21. Further, the saponin compounds provided may be used as chemical probe in photoaffinity labeling (PAL) experiments since they bear an acyl chain comprising a photoreactive moiety selected from diazirine, benzophenone and azido as well as a terminal biotin that can be used as identification/reporter tag.

One aspect of the present invention relates to a compound of general formula (I) or a pharmaceutically acceptable salt thereof

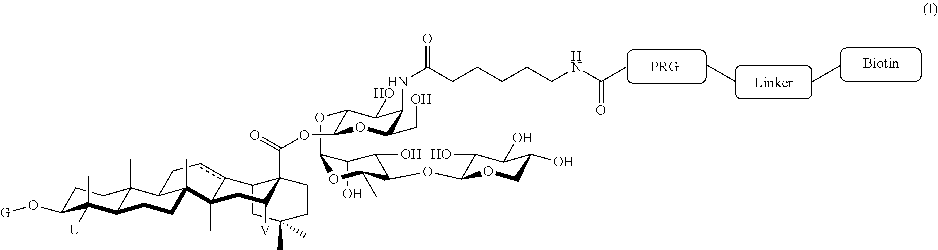

wherein G is hydrogen, a branched trisacchride of formula (VI) or a stereoisomer of formula (VI)

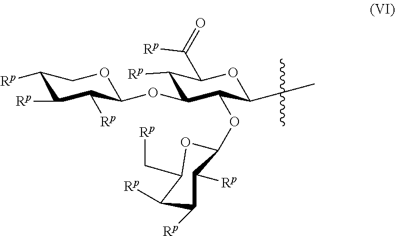

wherein each occurrence of RP is independently hydrogen or OR^{q};
wherein each occurrence of R^{q} is independently hydrogen or an optionally substituted group selected from 6-10-membered aryl, benzyl, C₁₋₆ aliphatic, or C₁₋₆ heteroaliphatic having 1-2 heteroatoms independently selected from the group consisting of nitrogen, oxygen, and sulfur; or two R^{q} are taken together to form a 5-7-membered heterocyclic ring having 1-2 heteroatoms independently selected from the group consisting of nitrogen, oxygen, and sulfur;
[symbol] is a single or double bond;
U is —CH₃, —CH₂—OH, —C═O or —CH═NOH;
V is H or OR^{x};
wherein R^{x} is independently hydrogen or an oxygen protecting group selected from the group consisting of alkyl ethers, benzyl ethers, silyl ethers, acetals, ketals, esters, carbamates, and carbonates;
PRG is a moiety comprising a photoreactive group selected from diazirine, benzophenone and azido or a combination thereof;
Linker is a chemical spacer; and
Biotin is a biotin moiety.

Another aspect of the present invention relates to a method of synthesizing a compound of general formula (I) or a pharmaceutically acceptable salt thereof which comprises reacting:

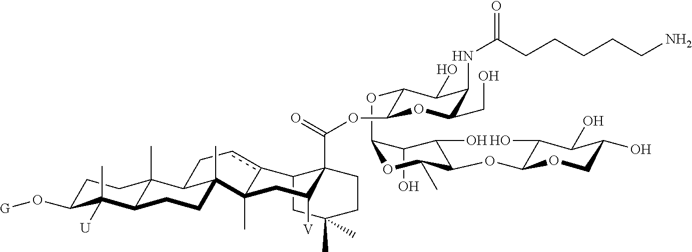

wherein G, [symbol] , U and V take the meanings as previously defined in formula (I);
with

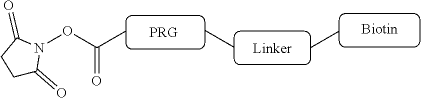

wherein PRG, linker and biotin take the meanings as previously defined in formula (I).

Another aspect of the present invention relates to a pharmaceutical composition comprising a compound of general formula (I) or a pharmaceutically acceptable salt thereof, a pharmaceutically acceptable carrier and an antigen.

Another aspect of the present invention relates to a compound of general formula (I) or a pharmaceutically acceptable salt thereof for use in medicine.

Another aspect of the present invention relates to a compound of general formula (I) or a pharmaceutically acceptable salt thereof or a pharmaceutical composition thereof for use in the treatment and/or prevention of cancer, an infectious disease or a neurodegenerative disease.

Another aspect of the present invention relates to a compound of general formula (I) or a pharmaceutically acceptable salt thereof for use as chemical probe in photoaffinity labeling (PAL) experiments.

These aspects and preferred embodiments thereof are additionally also defined hereinafter in the detailed description and in the claims.

### BRIEF DESCRIPTION OF THE FIGURES

To better understand the invention, its objects and advantages, the following figures are attached to the specification in which the following is depicted:

FIG. 1 shows the toxicity assessment in mice by median weight loss after injection of OVA or one of adjuvants tested: QS-21, BTS-25-BP and BTS-31-DAz.

FIG. 2 shows antibody responses in mice induced by adjuvant BTS-31-DAz upon coadministration with OVA antigen. (A-B) ELISA standard curves showing total anti-OVA IgG levels (represented as optical density [OD] measurements) in mouse sera on (A) day 21 and (B) day 35. (C-D) Anti-OVA total IgG titers on (C) day 21 and (D) day 35. Data points correspond to individual mice (five animals per group) and horizontal bars indicate median titers. Statistical significance was assessed by comparing each group to the no-adjuvant (OVA alone) control using a two-tailed unpaired Student's t-test with a 95% confidence interval (CI) *p≤0.05, **p≤0.01, ***p≤0.001.

FIG. 3 shows IgG subtyping of anti-OVA antibodies at day 35 resulting from mouse vaccinations with adjuvant BTS-31-DAz coadministered with OVA antigen. (A-C) ELISA standard curves showing anti-OVA IgG subtype levels (represented as optical density [OD] measurements) of (A) IgG1, (B) IgG2b, and (C) IgG2c (d) IgG3 antibodies on day 28 after first immunization. Statistical significance across the different dose-response curves was assessed by comparing to the no-adjuvant (OVA alone) control group using two-way ANOVA Dunnett's multiple comparisons test at the various dilutions. *p≤0.05, **p≤0.01, ***p≤0.001, ****p≤0.0001.

### DETAILED DESCRIPTION OF THE INVENTION

The clinical success of anticancer, antiviral, and antimicrobial vaccines critically depends on the identification of, and access to, novel potent adluvants with attenuated toxicity. In this context, specific fractions from extracts of the bark of Quillaja saponaria (QS) have proven to be exceedingly powerful adjuvants in immunotherapy. The QS-21 fraction is one of the most potent adjuvants known. QS-21 stimulates both antibody-based humoral immune responses (Th2) and cellular immunity (Th1), including production of antigen-specific cytotoxic T-lymphocytes. Vaccines containing QS-21, either alone in purified form or as a major component of adjuvant mixtures (e.g., Quil A, ISCOMs, ISCOMATRIX, AS01, AS02), have been investigated in clinical trials for cancers (melanoma, sarcoma, breast, prostate, ovarian, lung), infectious diseases (hepatitis, HIV, malaria, tuberculosis) and Alzheimer's disease.

Despite its remarkable potency and extensive clinical investigation, which has resulted in its approval as part of recently licensed vaccines against malaria and shingles disease, QS-21 suffers from several limitations. First, access to homogeneous QS-21 is limited due to an exceedingly low-yielding isolation and heterogeneity of crude extracts from Quillaja saponaria. Second, QS-21 is associated with clinical toxicity including swelling and erythema at the injection site, and systemic flu-like symptoms. Third, QS-21 undergoes spontaneous hydrolysis of the acyl chain domain ester linkages, producing adjuvant-inactive and hemolytic byproducts, complicating formulation and storage. Finally, the mechanisms of action of QS-21 are poorly understood, hindering rational design of improved variants and optimal matching of adjuvants with vaccine antigens based on desired immunological end points. The inherent liabilities of QS-21 highlight the need for improved analogues.

After extensive research, the present inventors have unexpectedly found new saponin compounds which bear a modified acyl chain comprising a photoactivatable group linked to a biotin tag through a spacer. These compounds act as potent adjuvants when co-administered with antigens, inducing an antibody response significantly higher than QS-21 and/or with lower toxicity.

Further, the compounds of the invention may be suitable for carrying out photoaffinity labeling (PAL) experiments, which is a frequently used tool in drug discovery for identifying new drug targets and molecular interactions, and for probing the location and structure of binding sites.

Unless defined otherwise, all technical and scientific terms and expressions used herein have the same meaning as commonly understood to one of ordinary skill in the art to which this disclosure belongs.

The term “aliphatic” or “aliphatic group” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not cyclic (also referred to herein as “carbocycle,” “cycloaliphatic” or “cycloalkyl”), that has a single point of attachment to the rest of the molecule. Unless otherwise specified, aliphatic groups contain 1-12 aliphatic carbon atoms. In some embodiments, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “acyl,” used alone or a part of a larger moiety, refers to groups formed by removing a hydroxy group from a carboxylic acid.

The term “halogen” means F, Cl, Br, or I.

The terms “aralkyl” and “arylalkyl” are used interchangeably and refer to alkyl groups (e.g. C₁₋₆ alkyl) in which a hydrogen atom has been replaced with an aryl group (e.g. 6-10-membered aryl). Such groups include, without limitation, benzyl, cinnamyl, and dihyrocinnamyl.

The term “aryl” used alone or as part of a larger moiety as in “aralkyl”, “aralkoxy”, or “aryloxyalkyl”, refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring”.

In certain embodiments of the present invention, “aryl” refers to an aromatic ring system which includes, but not limited to, phenyl, biphenyl, naphthyl, anthracyl and the like, which may bear one or more substituents. Also, included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-” used alone or as part of a larger moiety, e.g., “heteroaralkyl” or “heteroaralkoxy” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group” or “heteroaromatic” any of which terms include rings that are optionally substituted. The terms “heteroaralkyl” and “heteroarylalkyl” refer to an alkyl group substituted by a heteroaryl moiety, wherein the alkyl and heteroaryl portions independently are optionally substituted.

The term “heteroaliphatic” as used herein, means aliphatic groups wherein one or two carbon atoms are independently replaced by one or more of oxygen, sulfur, nitrogen, or phosphorus. Heteroaliphatic groups may be substituted or unsubstituted, branched or unbranched, cyclic or acyclic, and include “heterocycle”, “heterocyclyl”, “heterocycloaliphatic” or “heterocyclic” groups.

As used herein, the terms “heterocycle”, “heterocyclyl”, “heterocyclic radical” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety” and “heterocyclic radical” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined. In another aspect, the present invention provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration by injection.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary, tertiary, or quaternary amine. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Representative organic amines useful for the formation of base addition salts include ethylamine, diethylamine, ethylenediamine, ethanolamine, diethanolamine, piperazine and the like. (See, for example, Berge et al., supra).

Unless otherwise stated, structures depicted herein are also meant to include all isomeric (e.g., enantiomeric, diastereomeric, and geometric (or conformational)) forms of the structure; for example, the R and S configurations for each stereocenter, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Provided compounds may comprise one or more saccharide moieties. Unless otherwise specified, both D- and L-configurations, and mixtures thereof, are within the scope of the invention. Unless otherwise specified, both α- and β-linked embodiments, and mixtures thereof, are contemplated by the present invention.

If, for instance, a particular enantiomer of a compound of the present invention is desired, it may be prepared by asymmetric synthesis, chiral chromatography, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

One of ordinary skill in the art will appreciate that the synthetic methods, as described herein, utilize a variety of protecting groups. By the term “protecting group,” as used herein, it is meant that a particular functional moiety, e.g., O, S, or N, is masked or blocked, permitting, if desired, a reaction to be carried out selectively at another reactive site in a multifunctional compound. In preferred embodiments, a protecting group reacts selectively in good yield to give a protected substrate that is stable to the projected reactions; the protecting group is preferably selectively removable by readily available, preferably non-toxic reagents that do not attack the other functional groups; the protecting group forms a separable derivative (more preferably without the generation of new stereogenic centers); and the protecting group will preferably have a minimum of additional functionality to avoid further sites of reaction. As detailed herein, oxygen, sulfur, nitrogen, and carbon protecting groups may be utilized. Suitable carboxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. By way of non-limiting example, hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy) methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′ tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (lpaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′ phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonyl vinyl carbamate, o-(N,N-dimethylcarboxamido)benzyl carbamate, 1,1-dimethyl-3-(N,N-dimethylcarboxamido)propyl carbamate, 1, 1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)-amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fern), N-2-picolylamino N′ oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N,N-dimethylaminomethylene)amine, N′,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), p-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described by Greene and Wuts (supra).

As described herein, compounds of the invention may contain “optionally substituted” moieties. In general, the term “substituted” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph, which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)R^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched)alkylene)O—N(R^{∘})₂; or —(C₁₋₄ straight or branched)alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6-membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘} taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{Δ}, -(haloR^{Δ}), —(CH₂)₀₋₂OH, —(CH₂)₀₋₂OR^{Δ}, —(CH₂)₀₋₂CH(OR^{Δ})₂; —O(haloR^{Δ}), —CN, —N₃, —(CH₂)₀₋₂C(O)R^{Δ}, —(CH₂)₀₋₂C(O)OH, —(CH₂)₀₋₂C(O)OR^{Δ}, —(CH₂)₀₋₂SR^{Δ}, —(CH₂)₀₋₂SH, —(CH₂)₀₋₂NH₂, —(CH₂)₀₋₂NHR^{Δ}, —(CH₂)₀₋₂NR^{Δ}₂, —NO₂, —SiR^{Δ}₃, —OSiR^{Δ}₃, —C(O)SR^{Δ}, —(C₁₋₄straight or branched alkylene)C(O)OR^{Δ}, or —SSR. wherein each R^{Δ} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{Δ}, -(haloR^{Δ}), —OH, —OR^{A}, —O(haloR^{Δ}), —CN, —C(O)OH, —C(O)OR^{Δ}, —NH₂, —NHR^{Δ}, —NR^{Δ}₂, or —NO₂, wherein each R^{Δ} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur. Suitable substituents on the aliphatic group of R^{†} are independently halogen, —R^{Δ}, -(haloR^{Δ}), —OH, —OR^{Δ}, —O(haloR^{Δ}), —CN, —C(O)OH, —C(O)OR^{Δ}, —NH₂, —NHR^{Δ}, —NR^{Δ}₂, or —NO₂, wherein each R^{Δ} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration”, “administered systemically”, “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

“Liposomes” as used herein refer to closed bilayer membranes containing an entrapped aqueous volume. Liposomes may also be uni-lamellar vesicles possessing a single membrane bilayer or multi-lamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Liposomes, as they are ordinarily used, consist of smectic mesophases, and can consist of either phospholipid or nonphospholipid smectic mesophases. Smectic mesophase is most accurately described by Small, HANDBOOK OF LIPID RESEARCH, Vol. 4, Plenum, NY, 1986, pp. 49-50. According to Small, “[w]hen a given molecule is heated, instead of melting directly into an isotropic liquid, it may instead pass through intermediate states called mesophases or liquid crystals, characterized by residual order in some directions but by lack of order in others. . . . In general, the molecules of liquid crystals are somewhat longer than they are wide and have a polar or aromatic part somewhere along the length of the molecule. The molecular shape and the polar-polar, or aromatic, interaction permit the molecules to align in partially ordered arrays. . . . These structures characteristically occur in molecules that possess a polar group at one end. Liquid crystals with long-range order in the direction of the long axis of the molecule are called smectic, layered, or lamellar liquid crystals. . . . In the smectic states the molecules may be in single or double layers, normal or tilted to the plane of the layer, and with frozen or melted aliphatic chains.”

The term “enriched” as used herein refers to a mixture having an increased proportion of one or more species. In some embodiments, the mixture is “enriched” following a process that increases the proportion of one or more desired species in the mixture. In some embodiments, the desired species comprise(s) greater than 10% of the mixture. In some embodiments, the desired species comprise(s) greater than 25% of the mixture. In some embodiments, the desired species comprise(s) greater than 40% of the mixture. In some embodiments, the desired species comprise(s) greater than 60% of the mixture. In some embodiments, the desired species comprise(s) greater than 75% of the mixture. In some embodiments, the desired species comprise(s) greater than 85% of the mixture. In some embodiments, the desired species comprise(s) greater than 90% of the mixture. In some embodiments, the desired species comprise(s) greater than 95% of the mixture. Such proportions can be measured any number of ways, for example, as a molar ratio, volume to volume, or weight to weight.

The term “pure” refers to compounds that are substantially free of compounds of related non-target structure or chemical precursors (when chemically synthesized). This quality may be measured or expressed as “purity.” In some embodiments, a target compound has less than about 30%, 20%, 10%, 5%, 2%, 1%, 0.5%, and 0.1% of non-target structures or chemical precursors.

### Compounds of Formula (I)

In one aspect, the present application provides compounds of general formula (I) or a pharmaceutically acceptable salt thereof which are useful as adjuvants and as photoaffinity probes.

In some embodiments, the present application provides compounds of formula (I) or a pharmaceutically acceptable salt thereof
wherein G is hydrogen or

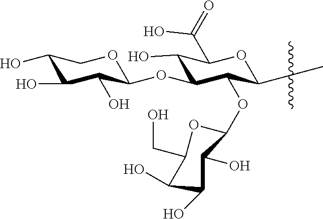

In certain embodiments, G is hydrogen. In certain embodiments, G is β-D-xylose

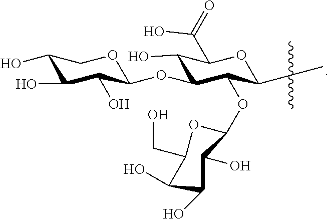

In certain embodiments, U is —CH₃. In certain embodiments, U is —CH₂—OH. In certain embodiments, U is —C═O. In certain embodiments, U is —CH═NOH.

In certain embodiments, V is H or OH. In certain embodiments, V is H. In certain embodiments, V is OH.

In certain embodiments, wherein G is hydrogen or or

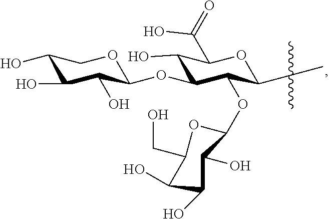

[symbol] is a single or double bond;
U is —C═O;
V is H or OR^{x}; and
R^{x} is independently hydrogen or an oxygen protecting group selected from the group consisting of alkyl ethers, benzyl ethers, silyl ethers, acetals, ketals, esters, carbamates, and carbonates.

In certain embodiments, wherein G is hydrogen or or

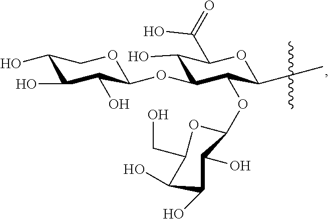

[symbol] is a single or double bond;
U is —CH₃, —CH₂—OH, —C═O or —CH═NOH;
V is H or OH.

In certain embodiments, G is hydrogen or

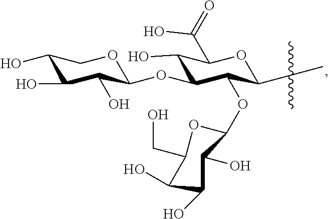

[symbol] is a single or double bond;
U is —C═O;
V is H or OH.

In certain embodiments, G is hydrogen or or

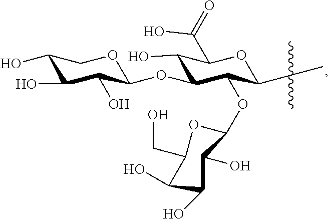

[symbol] is a single or double bond;
U is —C═O;
V is OH.

In certain embodiments, G is

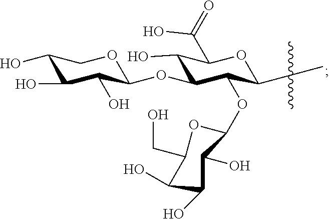

[symbol] is a single or double bond;
U is —C═O;
V is OH.

In certain embodiments, G is

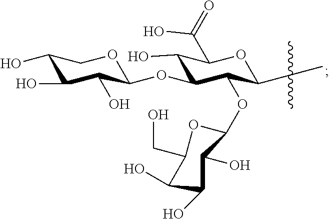

[symbol] is a double bond;
U is —C═O;
V is OH.

In general formula (I), PRG represents a moiety comprising a photoreactive group selected from diazirine, benzophenone and azido or a combination thereof. In more particular embodiments, PRG is a moiety comprising a photoreactive group selected from an aryldiazirine, a benzophenone, an arylzide or a combination thereof. In more particular embodiments, PRG is a moiety comprising a photoreactive group selected from a phenyldiazirine, a benzophenone, a phenylazide or a combination thereof.

In some embodiments, PRG is a moiety comprising a photoreactive group selected from diazirine including alkyl and aryl diazirines (e.g. phenyl diazirines) such as:

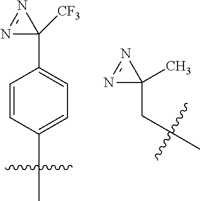

In some embodiments, PRG is a moiety comprising a photoreactive group selected from a benzophenone such as:

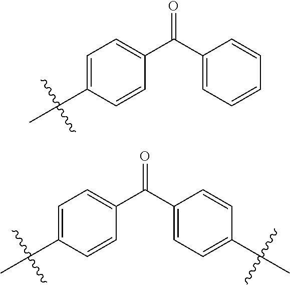 [SMILES: *c1ccc(C(=O)c2ccccc2)cc1 ; *c1ccc(C(=O)c2ccc(*)cc2)cc1]

In some embodiments, PRG is a moiety comprising a photoreactive group selected from azido, preferably an aryl azide such as a phenyl azide. In a more particular embodiment, such phenyl azide is:

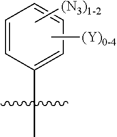

wherein Y is independently selected from halogen (e.g., Cl, F, Br, I), CH₃, OH, SH, NH₂, CN, CF₃, CCl₃, —CH₂—CH₃, —CH₂—OH, —CH₂NH₂, CH₂SH, CH₂Cl, CH₂Br, CH₂F, CHF₂, CH₂CN, CH₂CF₃, CH₂CCl₃, and CN. More particularly, Y is independently selected from F and CH₃.

Particular examples of phenyl azides include, but are not limited to:

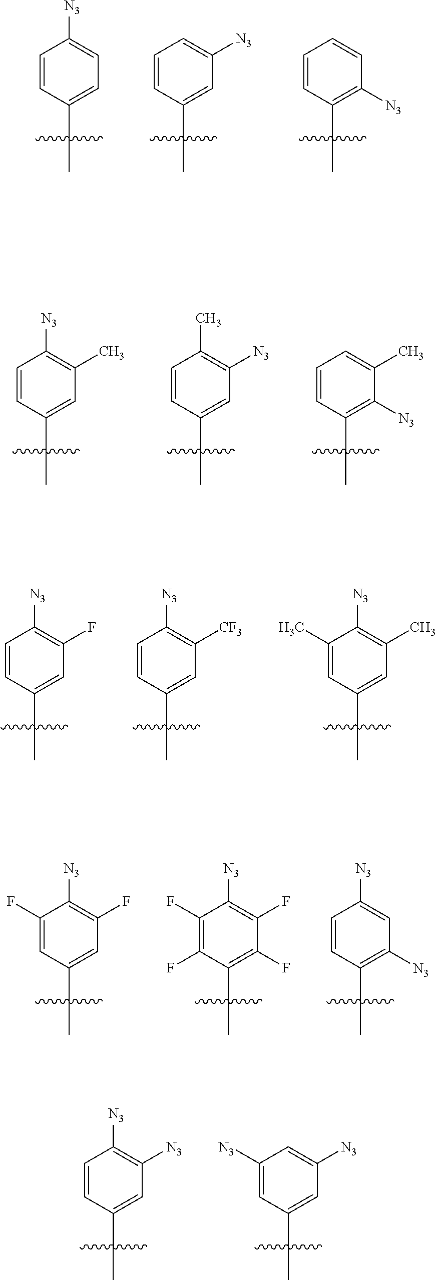

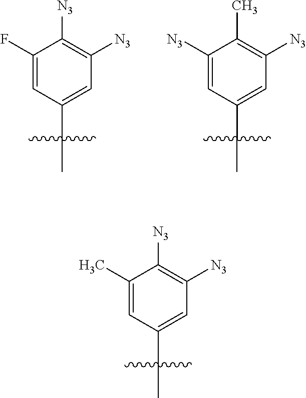 [SMILES: *c1cc(F)c(N=[N+]=[N-])c(N=[N+]=[N-])c1 ; *c1cc(N=[N+]=[N-])c(C)c(N=[N+]=[N-])c1 ; *c1cc(C)c(N=[N+]=[N-])c(N=[N+]=[N-])c1]

In general formula (I), linker represents a chemical spacer, that is, a carbon chain, which may include other elements, that covalently attaches PRG and Biotin. In a particular embodiment, the linker is or comprises (CH₂)₁₋₂₀ or is a divalent PEG-based spacer, preferably a divalent PEG-based spacer comprising formula

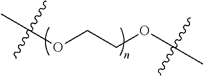

wherein n is a positive integer preferably selected from 1 to 20, more preferably 1 to 10 and even more preferably 1 to 5 such as 1, 2, 3, 4 or 5. In a particular embodiment n is 2, 3 or 4. In a more particular embodiment n is 2 or 4.

In general formula (I), biotin represents a biotin moiety, preferably.

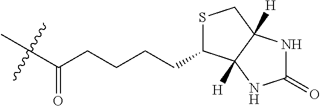

Exemplary compounds of general formula (I) are:

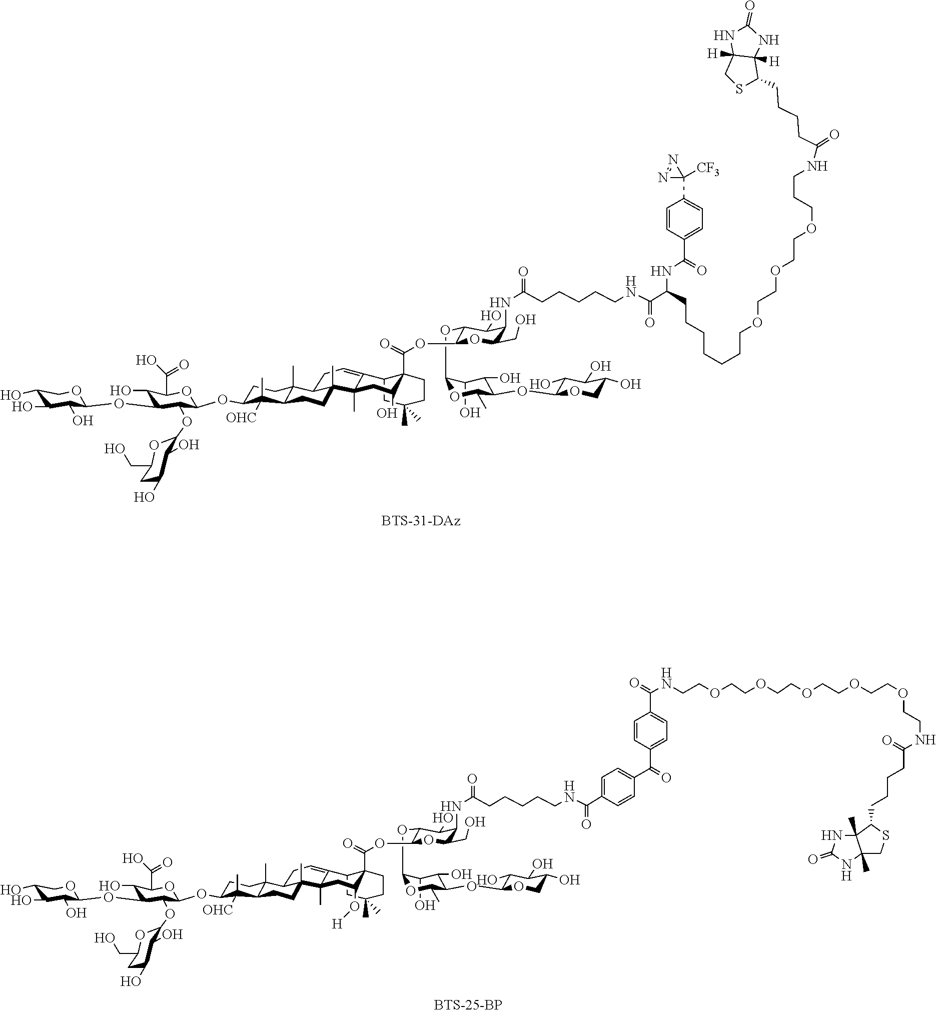

### Synthesis of Compounds

The compounds of formula (I) according to the present invention may be easily prepared through well-known transformations. By way of example, BTS-31-DAz and BTS-25-BP may be synthesized in total 30 and 27 steps starting from the semi-purified bark extract Quil A as shown in the experimental part.

Another aspect of the present invention relates to a method of synthesizing a compound of general formula (I) or a pharmaceutically acceptable salt thereof which comprises reacting:

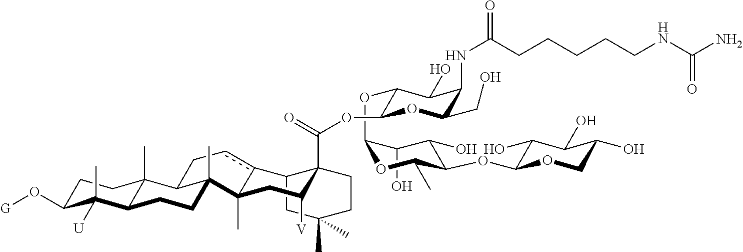

wherein G, [symbol] , U and V take the meanings as previously defined in formula (I);
with

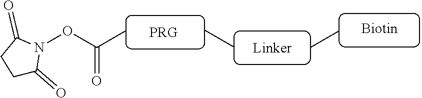

wherein PRG, linker and biotin take the meanings as previously defined in formula (I). Another aspect of the present invention relates to a method of synthesizing BTS-31-DAz or a pharmaceutically acceptable salt thereof which comprises at least one of the steps disclosed in the experimental part. In a particular embodiment, the method comprises reacting compounds 22 and 30.

Another aspect of the present invention relates to a method of synthesizing BTS-25-BP or a pharmaceutically acceptable salt thereof which comprises at least one of the steps disclosed in the experimental part. In a particular embodiment, the method comprises reacting compounds 22 and 24.

### Formulations

Another aspect of the present application relates to a formulation or pharmaceutical composition. The pharmaceutical composition comprises a compound or a pharmaceutically acceptable salt thereof according to the present invention such as BTS-31-DAz or BTS-25-BP, a pharmaceutically acceptable carrier and optionally an antigen.

In some embodiments, the pharmaceutical composition comprises one or both of BTS-31-DAz and BTS-25-BP or a pharmaceutically acceptable salt thereof, a pharmaceutically acceptable carrier and an antigen.

As used herein, the term “antigen” refers to a substance that is able to generate a specific immune response and induce the formation of specific antibodies or specially sensitized T cells or both. In the context of the present invention the antigen is therefore capable of activating lymphocytes, and as such, is a complete antigen, i.e., possesses antigenic properties de novo, being able to generate an immune response by themselves. Said antigen is characterized by a molecular mass above 14 kDa, having a complex chemical composition and ideally contains aromatic radicals. Said antigens belong to four main groups, proteins, polysaccharides, nucleic acids and lipids, preferably proteins.

An “immune response” to an antigen or immunogenic composition is the development in a subject of a humoral and/or a cell-mediated immune response to molecules present in the antigen or vaccine composition of interest. For purposes of the present invention, a “humoral immune response” is an antibody-mediated immune response and involves the induction and generation of antibodies that recognize and bind with some affinity for the antigen in the immunogenic composition of the invention, while a “cell-mediated immune response” is one mediated by T-cells and/or other white blood cells.

A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

The antigen may comprise at least one B cell epitope and/or a T cell epitope. As used herein, the term “B cell epitope” refers to any antigen portion or region that is recognized by secreted antibodies or B-cell receptors and is able to trigger an immune response in a B cell. Methods and techniques to determine if a peptide/molecule is or contains a B cell epitope are well known to the skilled person in the art and described in the literature (Ahmad, T. A., et al. Trials in Vaccinology 2016, Volume 5, Pages 71-83).

As used herein, the T cell epitope is selected from a helper T cell epitope or a CD8 epitope.

The term “Helper T cell epitope” as used herein refers to peptides derived from antigens and recognized by the Helper T-cell receptor (TCR) when bound to class II Major Histocompatibility Complex (MHC-II) molecules displayed on the cell surface of Antigen Presenting Cells (APCs), which lead to the activation of the Helper T cells. Said peptides are the specific amino acid sequence of the antigen which is recognized by the TCR. Said peptides can also be used as “helper epitopes”, epitopes which are known to be recognized by and activate Helper T cells, wherein said helper epitopes are fused to, or used in proximity of (such as co-expressed in the APC membrane), antigens of interest, more preferably B cell epitopes, being newly presented to Helper T cells in order to improve and enhance the immunological response of the Helper T cells. Methods to identify peptides which activate a response from helper T cells are well known in the art and examples of such methods can be found in the literature (Wang R. F. Methods. 2003, 29(3), 227-35; Singh R. et al. Immunology 2014, 141(4), 514-30).

The term “CD8 cell epitope” or “CD8 T cell epitope” as used herein refers to peptides derived from antigens recognized by CD8 T cell receptors when said antigens are bound to class I (MHC I) or class II (MHC II) Major Histocompatibility Complex (MHC) molecules on the surface of antigen presenting cells (APCs). When CD8 TCRs recognize the mentioned peptides, the cells get activated becoming cytotoxic T lymphocytes (CTLs). Methods to identify peptides which activate a response from CD8 T cells are well known in the art and examples of such methods can be found in the literature Reche, et al., 2004, Immunogenetics, vol. 56, 6, 405-419; Dönnes and Kohlbacher, 2005, Protein Sci., vol. 14, 8, 2132-2140; Doytchinova, et al., 2006, BMC Bioinformatics, vol. 7, 1, 131).

In some embodiments, the B cell epitope and/or the CD8 T cell epitope is selected from the group consisting of peptides, glycopeptides and carbohydrates capable of inducing an immune response against a neurodegenerative disease (e.g. the immunogenic region of a neurodegeneration-associated antigen), an infectious disease (e.g. the immunogenic region of a bacterial-, viral-, or protozoal-associated antigen) or a cancer cell (e.g. the immunogenic region of a cancer-associated antigen, also known as tumor-associated antigen or TAA).

Further examples of antigens useful in the present invention are, without limitation, tetanus toxoid, egg albumin, thyroglobulin, recombinant hemagglutinin B (rHagB) antigen, recombinant protein from H1N1 influenza, protective BpOmpW from Burkholderia pseudomallei.

In certain embodiments, the pharmaceutical composition includes a pharmaceutically acceptable amount of a compound of the present application. In certain embodiments, the pharmaceutical composition includes an immunologically effective amount of an antigen. In certain embodiments, the compounds of the application and an antigen form an active ingredient. In certain embodiments, the compound of the present application alone forms an active ingredient. The amount of active ingredient(s) which can be combined with a carrier material to produce a single dosage form will vary depending upon the host being treated, and the particular mode of administration. The amount of active ingredient(s) that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%, or from about 1% to 99%, preferably from 10% to 90%, 20% to 80%, 30% to 70%, 40% to 60%, 45% to 55%, or about 50%.

In certain embodiments, formulations of the present application include injectable formulations.

In one aspect the present application provides formulations comprising a liposome formulation of MPL and a compound of the present invention. In another aspect the present application provides formulations comprising MPL, a compound of the present invention and a squalene emulsion. In another aspect the present application provides formulations comprising MPL, a compound of the present invention, and CpG 7909 or CpG 1018. MPL is a heterogeneous mixture of molecules from a biological source including both agonists and antagonists for TLR4. CpG 7909 is an immunomodulating synthetic oligonucleotide designed to specifically agonise the Toll-like receptor 9 (TLR9).

In another aspect the present application provides formulations comprising immune stimulating complexes (ISCOM) or ISCOM matrices of a compound of the present invention. In another aspect the present application provides formulations comprising ISCOM matrices of a compound of the present invention and an antigen. ISCOMs are open cage-like nanoparticulate structures comprising a saponin (here, a compound of the present invention), cholesterol, phospholipid and an antigen. ISCOM particles are typically spherical of approximately 40 nm diameter. ISCOMs deliver antigen to the cytosol, and have been demonstrated to promote antibody response and induction of T helper cell as well as cytotoxic T lymphocyte responses in variety of experimental animal models. ISCOM matrices formulations (e.g. ISCOMATRIX and Matrix-M) contain the same components and have the same structure as the ISCOM but without the incorporated antigen.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Non-limiting examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid. ethylenediamine tetraacetic acid (EDTA), sorbitol, tartaric acid, phosphoric acid, and the like.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Non-limiting examples of suitable aqueous and nonaqueous carriers, which may be employed in the pharmaceutical compositions of the present application include water, alcohols (including but not limited to methanol, ethanol, butanol, etc.), polyols (including but not limited to glycerol, propylene glycol, polyethylene glycol, etc.), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain additives such as preservatives, wetting agents, emulsifying agents and dispersing agents. Prevention of the action of microorganisms upon the subject compounds may be ensured by the inclusion of various antibacterial and antifungal agents, for example, paraben, chiorobufanoi, phenol sorbic acid, and the like, it may also be desirable to include isotonic agents, such as sugars, sodium chloride, and the like into the compositions, in addition, prolonged absorption of the injectable pharmaceutical form may be brought about by the inclusion of agents which delay absorption such as aluminum monostearate and gelatin.

In some cases, in order to prolong the effect of a formulation, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form.

Regardless of the route of administration selected, the compounds of the present application, which may be used in a suitable hydrated form, and/or the pharmaceutical compositions of the present application, are formulated into pharmaceutically acceptable dosage forms by conventional methods known to those of skill in the art.

Actual dosage levels of the active ingredients in the pharmaceutical compositions of the present application may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present application employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the present application employed in the pharmaceutical composition at levels lower than that required to achieve the desired therapeutic effect and then gradually increasing the dosage until the desired effect is achieved.

In some embodiments, a compound or pharmaceutical composition of the present application is provided to a subject chronically. Chronic treatments include any form of repeated administration for an extended period of time, such as repeated administrations for one or more months, between a month and a year, one or more years, or longer. In many embodiments, a chronic treatment involves administering a compound or pharmaceutical composition of the present application repeatedly over the life of the subject. Preferred chronic treatments involve regular administrations, for example one or more times a day, one or more times a week, or one or more times a month, in general, a suitable dose, such as a daily dose of a compound of the present application, will be that amount of the compound that is the lowest dose effective to produce a therapeutic effect. Such an effective dose will generally depend upon the factors described above.

Generally, doses of the compounds of the present application for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used, in some embodiments, the dose administered to a subject may be modified as the physiology of the subject changes due to age, disease progression, weight, or other factors.

In some embodiments, provided adjuvant compounds of the present application are administered as pharmaceutical compositions or vaccines. In certain embodiments, it is contemplated that the amount of adjuvant compound administered will be 1-2000 μg. In certain embodiments, it is contemplated that the amount of adjuvant compound administered will be 1-1000 μg. In certain embodiments, it is contemplated that the amount of adjuvant compound administered will be 1-500 μg. in certain embodiments, it is contemplated that the amount of adjuvant compound administered will be 1-250 μg. In certain embodiments, it is contemplated that the amount of adjuvant compound administered will be 100-1000 μg. In certain embodiments, it is contemplated that the amount of adjuvant compound administered will be 100-500 μg. In certain embodiments, it is contemplated that the amount of adjuvant compound administered will be 100-200 μg. In certain embodiments, it is contemplated that the amount of adjuvant compound administered will be 250-500 μg. In certain embodiments, it is contemplated that the amount of adjuvant compound administered will be 10-1000 μg. In certain embodiments, it is contemplated that the amount of adjuvant compound administered will be 500-1000 μg. In certain embodiments, it is contemplated that the amount of adjuvant compound administered will be 50-250 μg. in certain embodiments, it is contemplated that the amount of adjuvant compound administered will be 50-500 μg.

In some embodiments, provided adjuvant compounds of the present application are administered as pharmaceutical compositions or vaccines. In certain embodiments, it is contemplated that the amount of adjuvant compound administered will be 1-2000 mg. In certain embodiments, it is contemplated that the amount of adjuvant compound administered will be 1-1000 mg. In certain embodiments, it is contemplated that the amount of adjuvant compound administered will be 1-500 mg. In certain embodiments, it is contemplated that the amount of adjuvant compound administered will be 1-250 mg. In certain embodiments, it is contemplated that the amount of adjuvant compound administered will be 100-1000 mg. In certain embodiments, it is contemplated that the amount of adjuvant compound administered will be 100-500 mg. In certain embodiments, it is contemplated that the amount of adjuvant compound administered will be 100-200 mg. In certain embodiments, it is contemplated that the amount of adjuvant compound administered will be 250-500 mg. In certain embodiments, it is contemplated that the amount of adjuvant compound administered will be 10-1000 mg. In certain embodiments, it is contemplated that the amount of adjuvant compound administered will be 500-1000 mg. In certain embodiments, it is contemplated that the amount of adjuvant compound administered will be 50-250 mg. In certain embodiments, it is contemplated that the amount of adjuvant compound administered will be 50-500 mg. In certain embodiments, it is contemplated that the amount of adjuvant compound administered will be 0.01-215.4 mg.

In certain embodiments, it is contemplated that the amount of adjuvant administered will be 1000-5000 μg/kg. In certain embodiments, it is contemplated that the amount of adjuvant administered will be 1000-4000 μg/kg. In certain embodiments, it is contemplated that the amount of adjuvant administered will be 1000-3000 μg/kg. In certain embodiments, it is contemplated that the amount of adjuvant administered will be 1000-2000 μg/kg. In certain embodiments, it is contemplated that the amount of adjuvant administered will be 2000-5000 μg/kg. In certain embodiments, it is contemplated that the amount of adjuvant administered will be 2000-4000 μg/kg. In certain embodiments, it is contemplated that the amount of adjuvant administered will be 2000-3000 μg/kg. In certain embodiments, it is contemplated that the amount of adjuvant administered will be 3000-5000 μg/kg. In certain embodiments, it is contemplated that the amount of adjuvant administered will be 3000-4000 μg/kg. In certain embodiments, it is contemplated that the amount of adjuvant administered will be 4000-5000 μg/kg. In certain embodiments, it is contemplated that the amount of adjuvant administered will be 1-500 μg/kg. In certain embodiments, it is contemplated that the amount of adjuvant administered will be 500-1000 μg/kg. In certain embodiments, it is contemplated that the amount of adjuvant administered will be 1000-1500 μg/kg. In certain embodiments, it is contemplated that the amount of adjuvant administered will be 1 mg/kg. In certain embodiments, it is contemplated that the amount of adjuvant administered will be 2 mg/kg. In certain embodiments, it is contemplated that the amount of adjuvant administered will be 3 mg/kg. In certain embodiments, it is contemplated that the amount of adjuvant administered will be 4 mg/kg. In certain embodiments, it is contemplated that the amount of adjuvant administered will be 5 mg/kg. In certain embodiments, it is contemplated that the amount of adjuvant administered will be 0.0029-5 mg/kg. In certain embodiments, the amount of adjuvant administered in females is less than the amount of adjuvant administered in males. In certain embodiments, the amount of adjuvant administered to infants is less than the amount of adjuvant administered to adults. In certain embodiments, the amount of adjuvant administered to pediatric recipients is less than the amount of adjuvant administered to adults. In certain embodiments, the amount of adjuvant administered to immunocompromised recipients is more than the amount of adjuvant administered to healthy recipients. In certain embodiments, the amount of adjuvant administered to elderly recipients is more than the amount of adjuvant administered to non-elderly recipients.

If desired, the effective dose of the active compound may be administered as two, three, four, five, six or more sub-doses administered separately at appropriate intervals throughout the day, optionally, in unit dosage forms. While it is possible for a compound of the present application to be administered alone, in certain embodiments the compound is administered as a pharmaceutical formulation or composition as described above.

The compounds according to the present application may be formulated for administration in any convenient way for use in human or veterinary medicine, by analogy with other pharmaceuticals.

The present application provides kits comprising pharmaceutical formulations or compositions of a compound of the present application. In certain embodiments, such kits include the combination of a compound of formula (I) such as BTS-31-DAz and BTS-25-BP and an antigen. The agents may be packaged separately or together. The kit optionally includes instructions for prescribing the medication, in certain embodiments, the kit includes multiple doses of each agent. The kit may include sufficient quantities of each component to treat one or more subject for a week, two weeks, three weeks, four weeks, or multiple months. The kit may include a full cycle of immunotherapy. In some embodiments, the kit includes a vaccine comprising one or more bacterial-, viral-, protozoal-, neurodegenerative disease- or cancer-associated antigens, and one or more provided compounds.

### Uses and Methods of Use of the Compounds of the Invention: Adjuvants and Probes

Compounds of formula (I) such as BTS-31-DAz and BTS-25-BP or a pharmaceutically acceptable salt thereof may be used as adjuvant in vaccines to increase the immune response to an antigen or enhance certain activities of cells from the immune system.

Another aspect of the present application relates to a compound of formula (I) or a pharmaceutically acceptable salt thereof for use in medicine, and more particularly, for use in the treatment and/or prevention of cancer, an infectious disease or a neurodegenerative disease.

It will be understood that the use a compound of formula (I) or a pharmaceutically acceptable salt thereof in therapy requires that the compound is administered in combination with the antigen to which a response is desired. In a preferred embodiment, the compound of formula (I) or a pharmaceutically acceptable salt thereof can be formulated with the antigen into an immunogenic composition. In this case, any reference to the medical use of a compound of formula (I) or a pharmaceutically acceptable salt thereof has to be understood as a reference to medical use of an immunogenic composition comprising a compound of formula (I) and an antigen. Similarly, any reference to a pharmaceutical composition of a compound of formula (I) or a pharmaceutically acceptable salt thereof has to be understood as a composition comprising an antigen.

Another aspect of the present application relates to a method for the treatment and/or prevention of a disorder in a subject said method comprising the administration of an effective amount of a compound of formula (I) or a pharmaceutically acceptable salt thereof, or a pharmaceutical composition thereof to the subject, wherein the disorder is cancer, an infectious disease or a neurodegenerative disease.

Another aspect of the present application relates to a compound of formula (I) or a pharmaceutically acceptable salt thereof, or a pharmaceutical composition thereof for use in the immunization of a subject.

Another aspect of the present application relates to a method for immunizing a subject, said method comprising administering to the subject an effective amount of a compound of formula (I) or a pharmaceutically acceptable salt thereof, or a pharmaceutical composition thereof.

Any animal that may experience the beneficial effects of the compositions of the present application is within the scope of subjects that may be treated, in some embodiments, the subjects are mammals. In some embodiments, the subjects are humans.

The vaccines of the present application may be used to confer resistance to infection by either passive or active immunization. When the vaccines of the present application are used to confer resistance through active immunization, a vaccine of the present application is administered to an animal to elicit a protective immune response which either prevents or attenuates a proliferative or infectious disease. When the vaccines of the present application are used to confer resistance to infection through passive immunization, the vaccine is provided to a host animal (e.g., human, dog, or mouse), and the antisera elicited by this vaccine is recovered and directly provided to a recipient suspected of having an infection or disease or exposed to a causative organism.

The present application thus concerns and provides a means for preventing or attenuating a proliferative disease resulting from organisms or tumor cells which have antigens that are recognized and bound by antisera produced in response to the immunogenic antigens included in vaccines of the present application. As used herein, a vaccine is said to prevent or attenuate a disease if its administration to an animal results either in the total or partial attenuation (i.e., suppression) of a symptom or condition of the disease, or in the total or partial immunity of the animal to the disease.

The administration of the vaccine (or the antisera which it elicits) may be for either a “prophylactic” or “therapeutic” purpose. When provided prophylacticaily, the vaccine(s) are provided in advance of any symptoms of proliferative disease. The prophylactic administration of the vaccine(s) serves to prevent or attenuate any subsequent presentation of the disease. When provided therapeutically, the vaccine(s) is provided upon or after the detection of symptoms which indicate that an animal may be infected with a pathogen or have a certain cancer. The therapeutic administration of the vaccine(s) serves to attenuate any actual disease presentation. Thus, the vaccines may be provided either prior to the onset of disease proliferation (so as to prevent or attenuate an anticipated infection or cancer) or after the initiation of an actual proliferation.

Thus, in one aspect the present application provides vaccines comprising one or more antigens (e.g. one or more bacterial, viral, protozoal, neurodegenerative disease or tumor-related antigens) in combination with a compound of formula (I) or a pharmaceutically acceptable salt thereof. In some embodiments, the vaccine comprises a single bacterial, viral, protozoal, neurodegenerative disease or tumor-related antigen in combination with a compound of formula (I) or a pharmaceutically acceptable salt thereof. In some embodiments, the vaccine comprises two or more bacterial, viral, protozoal, neurodegenerative disease or tumor-related antigens in combination with a compound of formula (I) or a pharmaceutically acceptable salt thereof.

In some embodiments, one or more antigens of provided vaccines are bacterial-associated antigens.

In certain embodiments, one or more antigens of provided vaccines are viral-associated antigens.

In certain embodiments, one or more antigens of provided vaccines are protozoal-associated antigens.

In certain embodiments, one or more antigens of provided vaccines are neurodegenerative disease-associated antigens.

In certain embodiments, one or more antigens of provided vaccines are cancer- or tumor-associated antigens.

One of ordinary skill in the art will appreciate that vaccines may optionally include a pharmaceutically acceptable excipient or carrier. Thus, according to another aspect, provided vaccines may comprise one or more antigens that are optionally conjugated to a pharmaceutically acceptable excipient or carrier. In some embodiments, said one or more antigens are conjugated covalently to a pharmaceutically acceptable excipient. In other embodiments, said one or more antigens are non-covalently associated with a pharmaceutically acceptable excipient.

As described above, adjuvants may be used to increase the immune response to an antigen. According to the present application, provided vaccines may be used to invoke an immune response when administered to a subject. In certain embodiments, an immune response to an antigen may be potentiated by administering to a subject a provided vaccine in an effective amount to potentiate the immune response of said subject to said antigen.

As described above, the compounds of the present application may be used in cancer vaccines as adjuvants in combination with tumor-associated antigens such as those listed hereinbefore. In certain embodiments, said vaccines may be used in the treatment or prevention of neoplasms. In certain embodiments, the neoplasm is a benign neoplasm. In other embodiments, the neoplasm is a malignant neoplasm. Any cancer may be treated using compounds of the invention with an antigen.

In certain embodiments, the malignancy is a hematological malignancy.

Other cancers besides hematological malignancies may also be treated using a compound of formula (I) or a pharmaceutically acceptable salt thereof. In certain embodiments, the cancer is a solid tumor.

In certain embodiments, compounds and pharmaceutical compositions of the present application can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (e.g., an inventive compound may be administered concurrently with another antiproliferative agent), or they may achieve different effects (e.g., control of any adverse effects).

For example, other therapies or anticancer agents that may be used in combination with the inventive anticancer agents of the present application include surgery, radiotherapy (gamma-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. Additionally, the present invention also encompasses the use of certain cytotoxic or anticancer agents currently in clinical trials and which may ultimately be approved by the FDA (including, but not limited to, epothilones and analogues thereof and geldanamycins and analogues thereof). For a more comprehensive discussion of updated cancer therapies see, www.nci.nih.gov, a list of the FDA approved oncology drugs at www.fda.gov/cder/cancer/druglistframe.htm, and The Merck Manual, Seventeenth Ed. 1999, the entire contents of which are hereby incorporated by reference.

In another aspect, the present application provides a method of treating infectious disease in a subject comprising administering to the subject a therapeutically effective amount of a compound of formula (I) or a pharmaceutically acceptable salt thereof and an antigen. In some embodiments, the infection is bacterial. In some embodiments, the infection is viral. In some embodiments, the infection is protozoal. Examples of infectious diseases include, but are not limited to, malaria, acquired immunodeficiency syndrome, hepatitis and tuberculosis. In some embodiments, the subject is human.

In another aspect, the present application provides a method of treating neurodegenerative diseases in a subject comprising administering to the subject a therapeutically effective amount of a compound of formula (I) or a pharmaceutically acceptable salt thereof and an antigen. In some embodiments, the present application can be used in the treatment of neurodegenerative diseases (e.g. Alzheimer's disease).

In another aspect, the present application provides a compound of formula (I) or a pharmaceutically acceptable salt thereof for use as chemical probe applying a chemical proteomics approach, which involves photoaffinity labeling (PAL) experiments for photocrosslinking followed by affinity purification and mass spectrometry (MS) analysis. PAL has become one of the most powerful strategies to study protein-protein interactions (PPIs) as well as to identify molecular targets of drugs or pharmaceutical agents for mechanistic elucidation. Upon photoirradiation, the photoreactive group (diazirine, benzophenone or azide) generates reactive intermediates to establish a covalent linkage with the protein of interest, whereas the biotin is used as a reporter group or tag for isolation/purification of the protein-drug covalent complex. In the case of target identification, the identity of the putative receptor is resolved using MS-based proteomic analysis.

It should be understood that the scope of the present disclosure includes all the possible combinations of embodiments disclosed herein.

The following examples are merely illustrative of certain embodiments of the invention and cannot be considered as restricting it in any way.

### EXAMPLES

### I. General Information

### Materials and Methods

All commercially available materials were used without further purification except boron trifluoride diethyl etherate and trifluoromethanesulfonic anhydride, which were distilled from calcium hydride and phosphorus pentoxide, respectively, at 1 atm under N₂. All manipulations with air-sensitive reagents and chemical reactions were carried out under a dry argon atmosphere using standard Schlenk techniques. Air- and moisture sensitive liquids and solutions were transferred via syringe. The appropriate carbohydrate reagents were dried via azeotropic removal of water with toluene. Molecular sieves were activated at 350° C. and were crushed immediately prior to use, then dried under vacuum. Organic solutions were concentrated under reduced pressure by rotary evaporation below 40° C. Column chromatography was performed employing 230-400 mesh silica gel. Thin-layer chromatography (TLC) was performed using aluminium-backed sheets precoated with 230-400 mesh silica gel 60 containing fluorescent indicator (F254). TLC plates were visualized under UV light (254 nm) and by staining with cerium ammonium molybdenate (CAM), phosphomolybdic acid (PMA), or 5% sulfuric acid in ethanol solutions or ninhydrin solutions.

### Nuclear Magnetic Resonance (NMR)

¹H, APT ¹³C, COSY and HSQC nuclear magnetic resonance. ¹H, APT ¹³C, COSY and HSQC spectra were recorded on a Bruker AVANCE NEO spectrometer (¹H NMR at 400 MHz and APT ¹³C NMR at 101 MHz), equipped with a SmartProbe and operating under TopSpin 4.1.1. Chemical shifts are expressed in parts per million (b scale) downfield from tetramethyl silane and are referenced to residual proton in the NMR solvent (CDCl₃: δ 7.26 for ¹H NMR, δ 77.00 for ¹³C NMR; methanol-d4: δ 3.31 for ¹H NMR, δ 49.15 for ¹³C NMR; CD₃CN:D₂O (3:7) δ 2.06 for ¹H NMR, δ 119.68 (—CN), δ 1.47 (—CD₃) for ¹³C NMR; D₂O: δ 1.96 for ¹H NMR, δ 118.26 (—CN), δ 1.79 (—CD₃) for ¹³C NMR). Data are presented as follows: chemical shift, multiplicity (s=singlet, br s=broad singlet, d=doublet, t=triplet, q=quartet, m=multiplet and/or multiple resonances), coupling constant in Hertz (Hz), integration, assignment.

### RP-HPLC Purification and LC-MS

All reverse-phase RP-HPLC analyses/purifications were carried out on a Waters 1525 binary gradient system (Solv. A=0.05% TFA in H₂O; Solv. B=0.05% TFA in CH₃CN) equipped with a Waters 2998 photodiode array detector (PDA), and combined with a low-resolution single quadrupole (SQD2, Waters Corporation) mass spectrometer. Absorbances were monitored at wavelengths of 190-600 nm.

### Immunological Evaluation in a Preclinical Mouse Vaccination Model

### Animals.

Animals were cared for and handled in compliance with the Guidelines for Accommodation and Care of Animals (European Convention for the Protection of Vertebrate Animals Used for Experimental and Other Scientific Purposes) and internal guidelines. Mice were housed in ventilated cages and fed on a standard diet ad libitum. All the experimental procedures were approved by the appropriate local authorities. The CIC bioGUNE animal facility is fully accredited by AAALAC International.

### Vaccination of Mice

Groups of five mice (C57BL/6, female, 6-8 weeks old) were vaccinated subcutaneously three times every 10 days (days 0, 14, and 28) with endotoxin-free OVA (EndoFit™ Ovalbumin, Invitrogen) (10 μg/mouse) in phosphate-buffered saline (PBS, 100 μL) either alone (no-adjuvant control group) or with the synthetic saponins (20 μg/mouse). To analyze the antibody responses over time, mice were bled via the submandibular vein at the indicated pre- (day −1) and post-vaccination timepoint (day 21), and by cardiac puncture at the experimental endpoint (day 35). Blood was collected in BD Microtainer® tubes (Clot Activator/SST™ Gel) and centrifuged at 7500 g for 10 min, after which serum was harvested and stored at −20° C. until further analysis.

### Evaluation of Immune Response

Analysis of the produced plasma antibodies specific against OVA were performed by an indirect enzyme-linked immunosorbent assay (ELISA). Briefly, Nunc MaxiSorp™ ELISA plates (Thermo Scientific) were coated with endotoxin-free OVA (EndoFit™ Ovalbumin; Invitrogen) at 0.05 μg/well in carbonate buffer (pH 9.5) and plates were incubated overnight at 4° C. After washing the wells (PBS, 10 mM, containing 0.05% Tween 20), plates were blocked with 10% of BSA (FCS, Biowest) in PBS buffer for 1 h. Serial dilutions of mouse sera in blocking buffer (10% FCS in PBS buffer) were added to wells with appropriate controls and incubated for 1 h at room temperature. After washing, goat anti-mouse total IgG (Jackson Immuno Research) or subclass specific IgG1, IgG2b, and IgG2c (Southern Biotech) antibodies conjugated to horseradish peroxidase (HRP) were added to each well. Antibodies were diluted as indicated by the manufacturer, i.e. IgG at 1/5000; IgG1 and IgG2b at 1/4000 dilution; and IgG2c at 1/10000. After 1 h incubation at room temperature, KPL SureBlue Reserve™ commercial solution (100 μl/well, SeraCare) containing 3,3′,5,5′-tetramethylbenzidine (TMB) was added as peroxidase substrate. The reaction was stopped after 10 min incubation by adding 2 N H₂SO₄ (100 μL/well). For absorbance measurements, optical density (OD) at 450 nm was immediately determined using a BioTek® Synergy HT multi-detection microplate reader. Antibody titer was defined as the highest serum dilution that showed an absorbance of 0.1 or greater over that of the pre-sera (1-3).

### Initial Toxicity Assessment in Mice

As a standard initial overall assessment of the potential toxicity of the saponins, in addition to visual inspection for potential signs of discomfort, the weight loss of each group of mice was monitored before and after each immunization. The median percentage weight change at different timepoints post-injection was determined and analyzed.

### Statistics

Two-way ANOVA Dunnett's multiple comparisons test was used to assess statistical significance across the different antibody response (OD) curves at the various dilutions compared to the positive control QS21 and saponin of interests i.e., either BTS-31-DAz or BTS-25-BP, or to the no-adjuvant, OVA-alone control. Antibody titers data defined as previously reported (Fernández-Tejada A. Pure Appl Chem (2017) 89:1359-78; Ghirardello, M. et al. Chem. Commun. 2020, 56 (5), 719-722; R. Fuentes, et al. Front. Immunol., 2022, 13, 1-11) are represented for each individual mouse; black horizontal bars indicate median value of five mice. When comparing two experimental groups, statistical significance of each antibody response compared to the no-adjuvant group was assessed in each case using a two-tailed unpaired Student's t-test with a 95% confidence interval (GraphPad Prism, GraphPad Software, La Jolla, CA). * p≤0.05, ** p≤0.01, *** p≤0.001, **** p≤0.0001.

### II. Synthetic Examples

### Chemical Synthesis of BTS-31-DAz

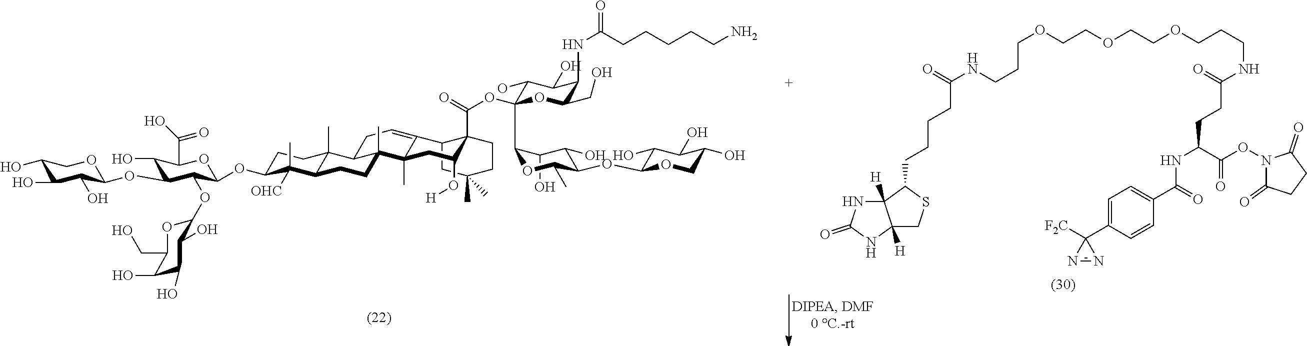

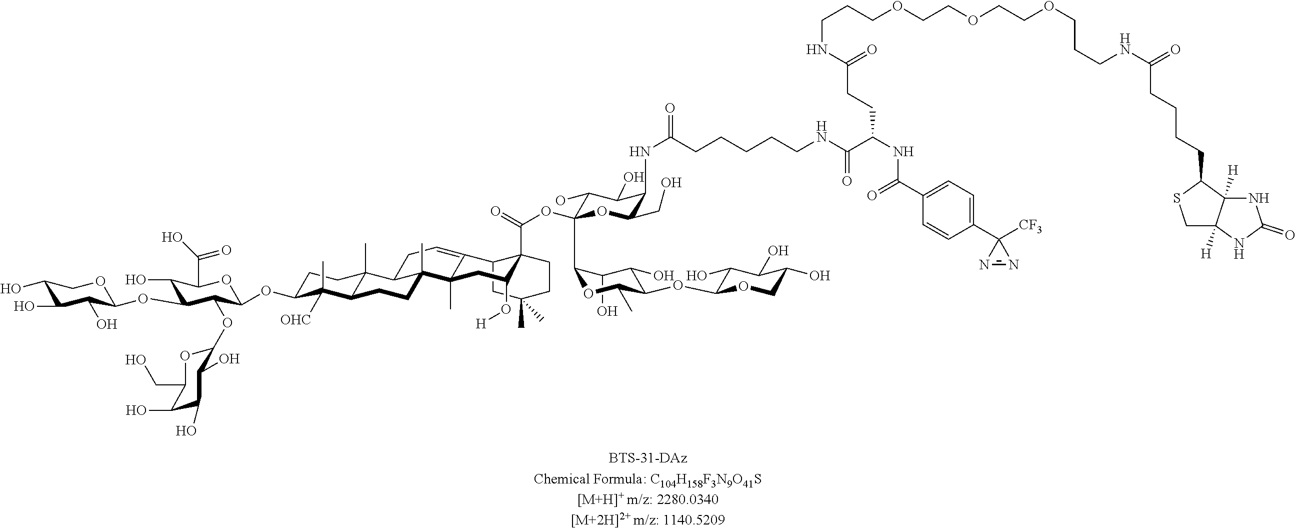

Synthesis of BTS-31-DAz: In a small vial, aminoacyl Saponin 22 (7.38 mg, 4.8 μmol, 1 eqv.), which was synthesized using previously reported protocol (Chea, E. K. et al. J. Am. Chem. Soc. 2012, 134, 13448-13457), was dissolved in 0.8 mL dry DMF and cooled to 0° C. To it, DIPEA (2 μL, 2.5 eqv.) was added and the mixture was stirred for 10-15 min. In another vial, previously HPLC purified lyophilized powder of NHS activated ester DAz-Glu(Biot)-NHS, 30 (6.4 mg, 7.3 μmol, 1.5 eqv.) was dissolved in 1.2 mL dry DMF at 0° C., which was then transferred to the saponin mixture via a gas tight syringe and stirred 0° C.-rt for 6 h. At 6 h, due to incomplete conversion an extra 1 μL of DIPEA was added to the mixture, stirred overnight. After 24 h, the crude was concentrated using high vacuum pump. The crude was dissolved in MeCN/Water=1:1 (6 mL) in ice bath condition and purified by RP-HPLC SemiPREP column using a linear gradient of 5→100% CH₃CN in water (0.05 vol % TFA) in 30 min. The fraction containing the major, single peak with t_{R}=20.19 min is collected and lyophilized to dryness to afford the Dazirine containing saponin, BTS-31-DAz (4.9 mg, 48%) as a white solid. The purity of the product was checked by C18 analytical column using a linear gradient of 5→100% CH₃CN in water (0.05 vol % TFA) in 30 min. The pure product appeared at t_{R}=17.66 min, λ₁ₘₐₓ=193.52 nm. λ₂ₘₐₓ=235. Characterization Data: ¹H NMR (400 MHz, CD₃CN:D₂O=30:70 v/v) δ 9.33 (s, 1H), 7.86 (d, J=8.2 Hz, 2H), 7.31 (d, J=8.4 Hz, 2H), 5.26 (s, 2H), 5.24 (s, 1H), 5.11 (s, 1H), 4.63 (d, J=7.7 Hz, 1H), 4.52 (d, J=7.8 Hz, 1H), 4.47-4.42 (m, 1H), 4.26 (dd, J=7.9, 4.5 Hz, 1H), 4.21 (s, 1H), 3.86 (s, 2H), 3.80-3.65 (m, 3H), 3.61 (t, J=9.5 Hz, 1H), 3.50 (dd, J=5.4, 2.6 Hz, 5H), 3.48-3.40 (m, 3H), 3.37 (t, J=6.3 Hz, 1H), 3.33-3.26 (m, 1H), 3.21-3.03 (m, 10H), 2.91-2.72 (m, 2H), 2.64 (d, J=12.9 Hz, 1H), 2.32-2.15 (m, 3H), 2.10 (t, J=7.4 Hz, 2H), 1.79 (s, 3H), 1.53 (ddq, J=58.3, 29.5, 7.3, 6.9 Hz, 6H), 1.32-1.13 (m, 9H), 1.05 (s, 2H), 0.97 (d, J=11.9 Hz, 1H), 0.88 (s, 2H), 0.81 (d, J=16.1 Hz, 5H), 0.63 (s, 3H). MS (ESI+) m/z: Calcd for [C₁₀₄H₁₅₉F₃N₉O₄₁S]⁺ [M+H]⁺ 2280.03, found: 2279.39.

(A) Synthesis of Fully Deprotected Saponin Scaffold with a Simple Acyl Chain, Compound 22:

The following steps were followed to synthesized fully deprotected acyl chain saponin 22:

### (i) Synthesis of Prosapogenins 1 & 2 (AS-II-002):

We started the semi-synthesis of the saponin BTS-31-DAz from the bark extract of Quillaja saponaria tree.

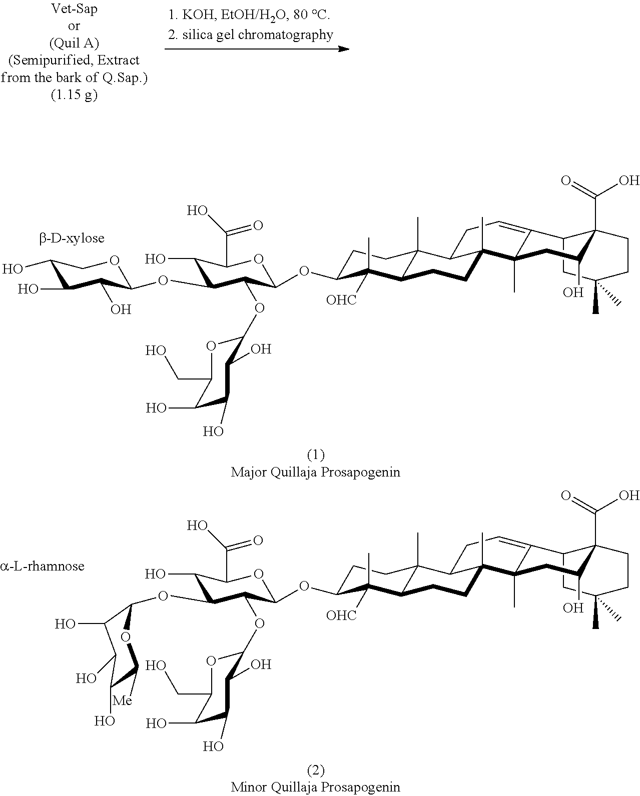

In a 250-mL round-bottom flask equipped with a reflux condenser, Vet-Sap/Quil A (1.15 g) and potassium hydroxide (0.97 g, 17 mmol) were suspended in ethanol: water (1:1) (50 mL), then the mixture is heated to 85° C. for 7 h. The reaction mixture was then cooled to 0° C., neutralized with 1.0 N HCl, and concentrated to approximately one-half of the volume. The crude was frozen and lyophilized, and the resulting dry solid is purified by silica gel chromatography (CH₂Cl₂/MeOH/Water/AcOH, 15:9:2:1). The major spot observed by TLC in the above solvent system is isolated by concentrating the fractions. The resulting solid is dried by azeotropic removal of solvents with toluene (2×20 mL) and lyophilized in MeCN/water (1:1) (3×15 mL) to provide a mixture of prosapogenins (1:2) as a light tan foam (~0.6 g, 50% mass yield). These, xylose and rhamnose-containing prosapogenins 1 and 2 respectively, correspond to the two most abundant trisaccharide-triterpene fragments found in QS saponins. R_{f}=0.71 (DCM/methanol/water/acetic acid 15:9:2:1); characteristic signals from 1H NMR: ¹H NMR (400 MHz, Methanol-d₄) δ 9.46 (s, 1H, —CHO), 5.39-5.29 (m, 1H, R2C═CHR), 5.13-4.97 (m, 1H), 4.81 (s, 1H), 4.61 (d, J=7.6 Hz, 1H), 4.46 (td, J=7.0, 6.0, 3.8 Hz, 2H), 4.34-4.10 (m, 2H), 4.09-4.01 (m, 2H), 4.01-3.34 (m, 18H), 3.32-3.16 (m, 4H), 3.02 (dd, J=14.4, 4.5 Hz, 1H), 2.61-2.47 (m, 1H), 2.47-2.37 (m, 1H), 2.37-2.25 (m, 1H), 2.03-1.86 (m, 6H), 1.86-1.67 (m, 4H), 1.67-1.58 (m, 2H), 1.58-1.50 (m, 2H), 1.50-1.30 (m, 7H), 1.30-1.19 (m, 4H), 1.17 (d, J=2.1 Hz, 4H), 1.12 (d, J=11.5 Hz, 1H), 1.09-0.84 (m, 13H), 0.81 (s, 2H).

### (ii) Synthesis of Triethylsilyl (TES) Protected Prosapogenin 3 by Selective Protection of Prosapogenin Hydroxyl Groups (AS-II-003):

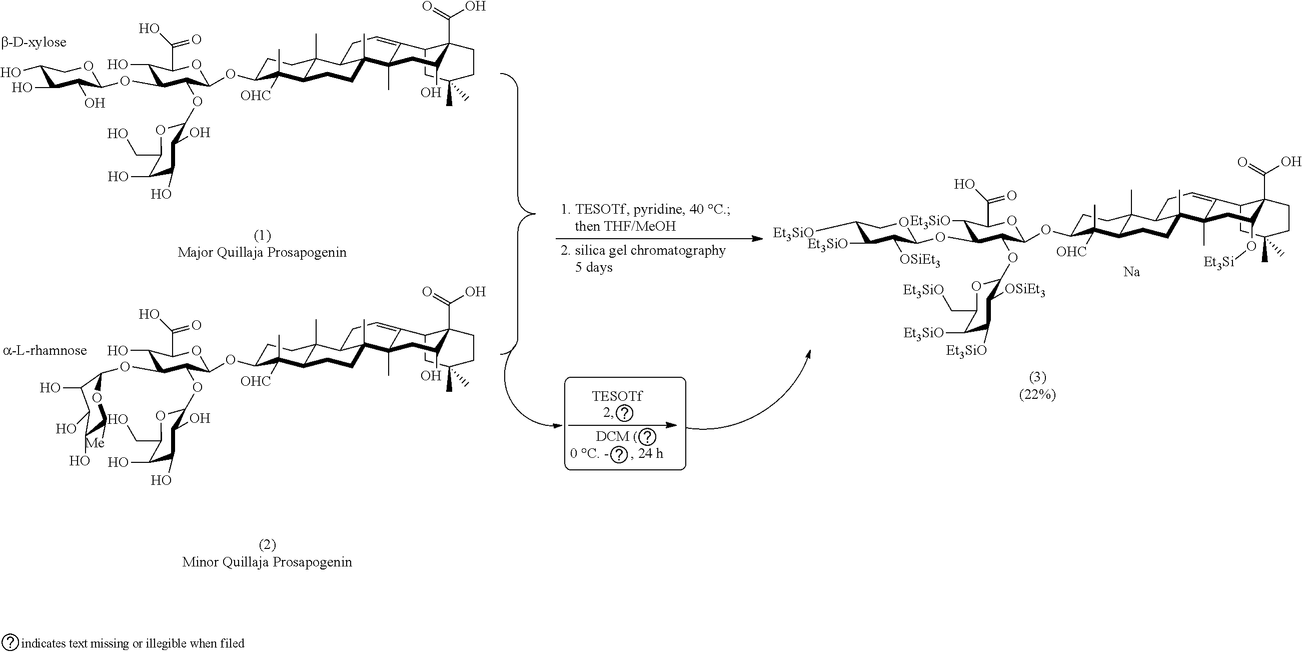

In a 250-mL round bottom flask, the solid mixture of prosapogenins 1 and 2 (~0.600 g) is azeotroped from pyridine (8 mL), kept in high vacuum for 1 h, then pyridine (8 mL) is added, followed by TESOTf (2.0 mL, 8.8 mmol) was added dropwise. The reaction mixture is stirred for 3 days, TLC indicated incomplete conversion then TESOTf (1 mL, 4.4 mmol) is added. On 5^{th} day, the mixture is concentrated and passed through a short plug of silica gel eluted with hexanes/EtOAc (4:1 to 2:1). The eluate is concentrated, the resulting yellow oil is dissolved in MeOH/THF (1:1) (20 mL), and the solution is stirred for another 3 days to remove the silyl esters by solvolysis.

The reaction mixture is concentrated and the resulting mixture of xylose- and rhamnose-containing (TES)₉-protected prosapogenin diacids (1 and 2) is separated by silica gel chromatography (hexanes/EtOAc, 4:1 to 2:1) to afford purified xylose-containing protected prosapogenin 3 (~0.25 g, ~21% yield) as a white solid.

Alternative route to synthesize 3 (AS-II-032): We developed an alternative method to synthesize compound 3 to avoid long time-consuming protocol as used earlier (shown in blue box). This new method shortened the time-consuming protocol from 8 days to 1-2 days only. Prosapoaenin 1 and 2 (~500 ma) was taken in 250 mL round bottom flask. Added dry DCM (50 mL) which gave a turbid solution; stirred and cooled to 0° C. To this suspension, 2,6-lutidine (10 eqv.) and then TESOTf (9 eqv.) was added slowly under Argon atmosphere. Stirred for 24 h and after that reaction was quenched by adding NaHCO₃ and extracted with DCM. Organic layer was concentrated and 2,6-lutidine in the crude was removed by high vacuum pump. Purification was done in a similar way as earlier affording 22% yield. R_{f}=0.61 (Hexane/ethyl acetate 4:2); ¹H NMR (400 MHz, Chloroform-d) δ 9.67 (s, 1H, —CHO), 5.33 (s, 1H, R₂C═CHR), 4.62-4.42 (m, 3H), 4.04-3.70 (m, 7H), 3.64 (q, J=8.2, 7.5 Hz, 2H), 3.57-3.32 (m, 4H), 3.28 (t, J=7.8 Hz, 1H), 3.12 (q, J=11.1 Hz, 1H), 3.03-2.90 (m, 1H), 2.23 (t, J=13.6 Hz, 1H), 1.99-1.58 (m, 9H), 1.37 (s, 3H, Me), 1.28 (s, 3H, Me), 0.99 (dq, J=17.3, 8.9, 8.4 Hz, 78H), 0.90 (s, 3H, Me), 0.68 (dp, J=25.0, 8.3, 7.7 Hz, 49H). ¹³C NMR (101 MHz, Chloroform-c) δ 211.32, 122.11, 101.54, 101.32, 79.38, 78.74, 76.13, 75.85, 75.63, 74.86, 72.45, 72.23, 70.98, 49.33, 46.27, 40.20, 32.67, 26.50, 24.27, 16.93, 15.78, 11.59, 7.44, 7.37, 7.24, 7.11, 7.04, 6.99, 6.91, 6.84, 6.77, 5.74, 5.51, 5.38, 5.26, 5.17, 5.09.

(iii) Synthesis of Protected Quillaja Prosapogenin 4 by Selective Esterification of Glucuronic Acid Carboxylic Acid in Protected Prosapogenin 3 (AS-II-034):

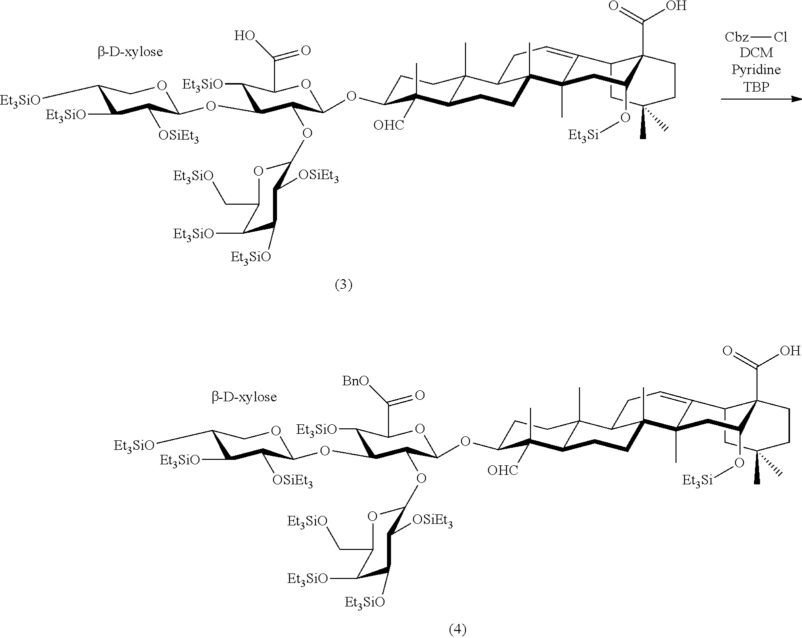

In a 10-mL modified Schlenk flask, the prosapogenin diacid 3 (250 mg, 0.126 mmol, 1.0 equiv.) is dissolved in dry DCM (2 mL), and added pyridine (202 μL, 2.52 mmol, 20 equiv.), TBP (623 mg, 2.52 mmol, 20 equiv.), and benzyl chloroformate (80 μL, 0.630 mmol, 5 equiv.). The mixture is stirred at rt overnight. After 18 h, complete conversion of the starting material is indicated by TLC. The crude is then concentrated and purified by silica gel chromatography (hexanes/EtOAc, 15:1 to 6:1) to afford selectively glucuronate-protected prosapogenin (4) (200 mg, 76%) as a white solid. R_{f}=0.39 (Hexane/ethyl acetate 7:1); ¹H NMR (400 MHz, Chloroform-d) δ 9.72 (s, 1H), 7.37 (d, J=9.9 Hz, 7H), 5.36 (d, J=4.1 Hz, 1H), 5.21 (dd, J=75.6, 12.2 Hz, 2H), 4.73 (s, 1H), 4.62-4.52 (m, 3H), 4.44 (d, J=7.2 Hz, 2H), 4.22 (t, J=7.3 Hz, 2H), 4.14 (q, J=7.2 Hz, 2H), 4.04-3.73 (m, 11H), 3.69-3.55 (m, 5H), 3.55-3.46 (m, 2H), 3.39 (td, J=14.0, 13.3, 7.4 Hz, 4H), 3.28 (t, J=8.0 Hz, 2H), 3.15 (t, J=10.9 Hz, 2H), 2.97 (dd, J=14.3, 4.3 Hz, 2H), 2.54 (s, 1H), 2.24 (t, J=13.5 Hz, 3H), 2.07 (s, 3H), 1.97-1.62 (m, 21H), 1.56 (d, J=12.6 Hz, 7H), 1.41 (d, J=14.2 Hz, 12H), 1.32 (s, 7H), 1.29 (d, J=5.8 Hz, 9H), 1.17 (dd, J=13.6, 6.9 Hz, 11H), 0.97 (ddd, J=28.5, 14.9, 7.1 Hz, 132H), 0.84-0.53 (m, 81H).

¹³C NMR (101 MHz, CDCl₃) δ 212.78, 182.78, 168.71, 157.62, 143.67, 135.63, 131.20, 129.13, 128.89, 128.80, 128.67, 128.61, 128.47, 127.98, 127.32, 122.45, 120.98, 103.81, 101.74, 101.20, 86.40, 79.21, 79.09, 77.68, 77.36, 77.04, 76.80, 76.44, 76.20, 75.46, 75.33, 72.98, 72.87, 71.78, 71.41, 67.17, 65.70, 60.75, 60.67, 54.24, 49.78, 49.08, 46.73, 46.51, 41.80, 40.53, 39.95, 38.27, 37.46, 36.47, 35.53, 34.98, 33.01, 32.69, 31.94, 31.90, 30.83, 30.05, 26.88, 25.66, 24.64, 23.63, 23.00, 21.37, 20.60, 17.17, 16.05, 14.53, 14.46, 12.48, 7.88, 7.79, 7.58, 7.47, 7.31, 7.18, 7.11, 6.27, 6.00, 5.81, 5.73, 5.70, 5.62, 5.59, 5.36, 5.07, 4.78.

(iv) Synthesis of Protected Aminogalactose Saponin 19 by Glycosylation of Prosapogenin 4 with Linear Trisaccharide 18 (AS-II-041):

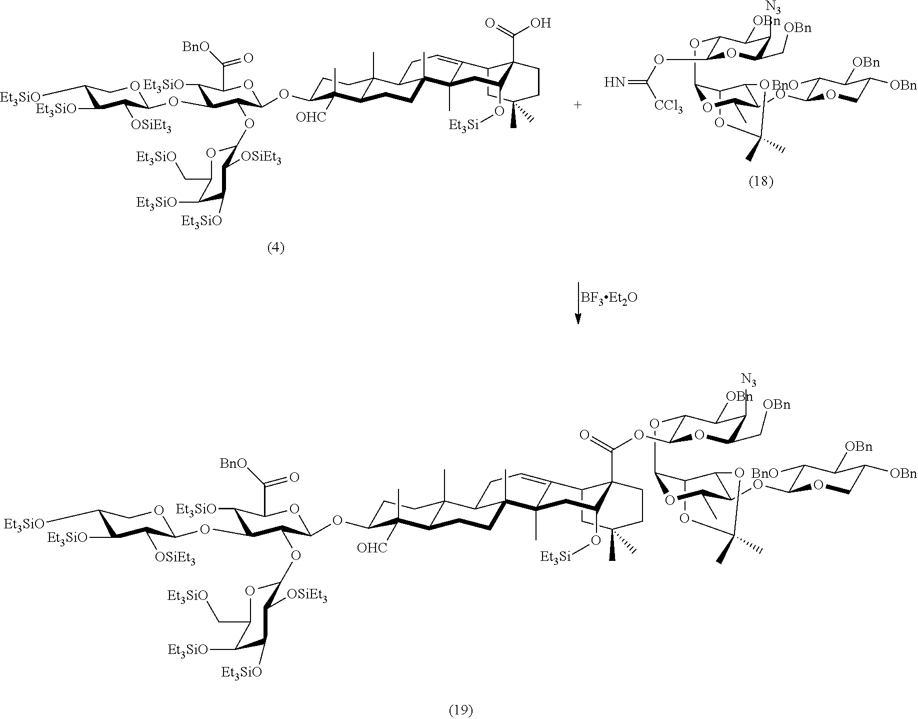

In a 25-mL modified Schlenk flask, the selectively protected prosapogenin 4 (222 mg, 0.107 mmol, 1.25 equiv.) and the linear trisaccharide imidate 18 which was synthesized using previously published protocol (Chea, E. K. et al. J. Am. Chem. Soc. 2012, 134, 13448-13457; Fernández-Tejada, A. et al Nat. Chem. 2014, 6 (7), 635-643) (100 mg, 0.0894 mmol, 1.0 equiv.) are azeotropically dried from toluene (3×1.5 mL) under high vacuum, then dissolved in dry DCM (5 mL). In another Schlenk flask, powdered 4 Å MS (500 g) is activated and then transferred to the above reaction mixture using hot funnel as fast as possible. The suspension is stirred for 30 min at rt. The opaque, white mixture is then cooled to −78° C. and BF₃·OEt₂ (12 μL, 0.0894 mmol, 1 equiv.) is injected via gas-tight syringe. The reaction mixture is stirred at −78° C. for 3 h, and then quenched by adding 0.5 mL TEA at −78° C., diluted with DCM and filtered. The crude is concentrated using rota vap. And purified by silica gel chromatography (hexane/EtOAc, 9:1 to 4:1 with 1% TEA) which afforded the prosapogenin-linear trisaccharide conjugate 19 (200 mg, 74%) as a glassy solid. R_{f}=0.56 (Hexane/ethyl acetate 8:2 with 1% triethylamine)¹H NMR (400 MHz, Benzene-d₆) δ 9.79 (s, 1H, —CHO), 7.58 (d, J=7.5 Hz, 3H), 7.43 (dd, J=12.5, 7.3 Hz, 6H), 7.33 (dq, J=11.3, 7.9, 5.9 Hz, 16H), 7.19 (ddd, J=18.8, 9.3, 6.8 Hz, 7H), 5.72 (d, J=7.9 Hz, 1H), 5.66 (d, J=4.6 Hz, 3H), 5.35-5.26 (m, 3H), 5.21 (d, J=11.6 Hz, 1H), 5.16-5.07 (m, 2H), 5.05-4.89 (m, 5H), 4.86 (d, J=7.2 Hz, 1H), 4.76 (d, J=6.9 Hz, 1H), 4.66-4.54 (m, 5H), 4.53-4.46 (m, 2H), 4.35 (ddd, J=27.1, 18.9, 11.8 Hz, 8H), 4.01-3.54 (m, 19H), 3.48-3.27 (m, 5H), 2.63 (t, J=13.5 Hz, 1H), 2.42-1.75 (m, 8H), 1.70 (d, J=6.9 Hz, 7H), 1.66-1.54 (m, 9H), 1.33 (dq, J=33.1, 8.3 Hz, 82H), 1.12 (ddt, J=53.0, 21.9, 7.0 Hz, 100H), 0.85 (tq, J=12.6, 7.9, 5.9 Hz, 25H), 0.59 (s, 5H), 0.39 (s, 1H).

### (v) Synthesis of Protected Aminogalactose Saponin 20 by Reduction of Protected Aminogalactose Saponin 19 (AS-II-042):

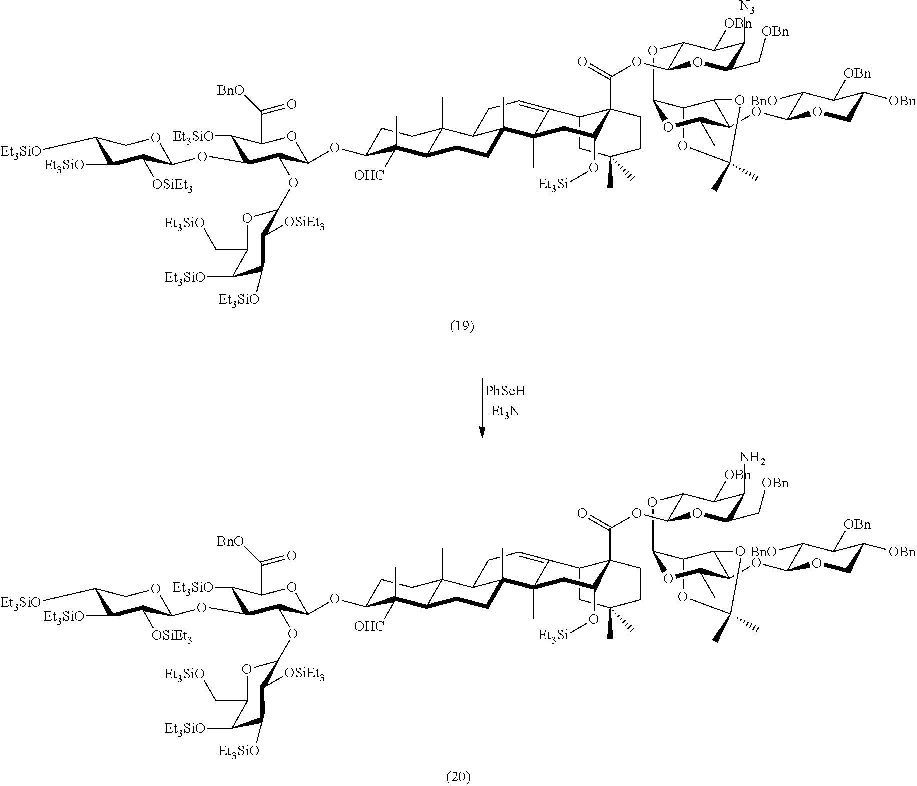

In a 25-mL modified Schlenk flask, PhSeSePh (205 mg, 0.66 mmol, 10 equiv.) is taken and passed Ar/vacuum cycle×3 and then dissolved in THE (5 mL) and H₃PO₂ (50% in water) (785 μL, 7.25 mmol, 110 equiv.). The yellow solution is heated at 40° C. for 1-2 h until it turns transparent. The reaction mixture is removed from the heat, diluted with toluene (5 mL) and distilled water (5 mL), and stirred vigorously for 5 min under Ar. The organic layer was transferred via a syringe to another flask containing anhydrous MgSO₄ under Ar atmosphere. This freshly prepared solution of PhSeH is then cannula transferred to a 50 mL Schlenk flask containing a solution of the azeotropically dried saponin azide 19 (200 mg, 0.0659 mmol, 1.0 equiv.) in dry Et₃N (30 mL). Upon addition, a white precipitate is formed, and the solution turns bright yellow. The reaction mixture is stirred for 4-5 h at 40° C., then concentrated to give a yellow-white solid. Purification by silica gel chromatography (started with toluene until nonpolar PhSeSePh has gone and then 98% toluene/EtOAc, to 90% toluene/EtOAc with 1 vol % Et₃N) to afford the saponin amine 20 (150 mg, 84%) as a glassy solid. R_{f}=0.38 (toluene/ethyl acetate 9:1 with 1% triethylamine) ¹H NMR (400 MHz, Benzene-d₆) δ 9.69 (s, 1H), 7.49-7.45 (m, 3H), 7.36-7.29 (m, 6H), 7.21 (dddd, J=11.6, 7.8, 6.4, 1.8 Hz, 17H), 7.13-7.05 (m, 7H), 5.69 (d, J=7.9 Hz, 1H), 5.60 (s, 1H), 5.56 (s, 1H), 5.23-5.16 (m, 3H), 5.10 (d, J=11.5 Hz, 1H), 5.03 (s, 1H), 5.01-4.97 (m, 2H), 4.94-4.73 (m, 7H), 4.66 (d, J=6.9 Hz, 1H), 4.55-4.46 (m, 5H), 4.45-4.34 (m, 5H), 4.27 (s, 6H), 4.23 (q, J=2.4, 1.9 Hz, 3H), 4.20 (d, J=1.8 Hz, 2H), 4.18 (s, 1H), 4.17-4.03 (m, 4H), 4.03-3.93 (m, 4H), 3.91-3.69 (m, 6H), 3.69-3.56 (m, 4H), 3.51 (ddd, J=13.7, 9.1, 6.5 Hz, 4H), 3.33 (dt, J=14.2, 7.3 Hz, 2H), 3.22 (dd, J=11.5, 9.5 Hz, 1H), 3.15 (dd, J=9.1, 4.0 Hz, 1H), 3.09 (dd, J=4.0, 1.6 Hz, 1H), 2.54 (t, J=13.5 Hz, 2H), 2.35-2.17 (m, 2H), 2.10 (s, 1H), 2.05-1.92 (m, 1H), 1.85 (s, 1H), 1.77 (d, J=2.3 Hz, 1H), 1.75-1.66 (m, 1H), 1.61 (d, J=6.0 Hz, 8H), 1.55 (s, 1H), 1.52 (s, 4H), 1.50 (d, J=2.8 Hz, 3H), 1.30-1.24 (m, 15H), 1.24-1.11 (m, 65H), 1.11-1.02 (m, 48H), 1.01-0.89 (m, 30H), 0.87 (s, 3H), 0.81-0.67 (m, 25H).

### (VI) Synthesis of Protected Aminoacyl Saponin 21 by Acylation of Protected Aminogalactose Saponin 20 (AS-II-044):

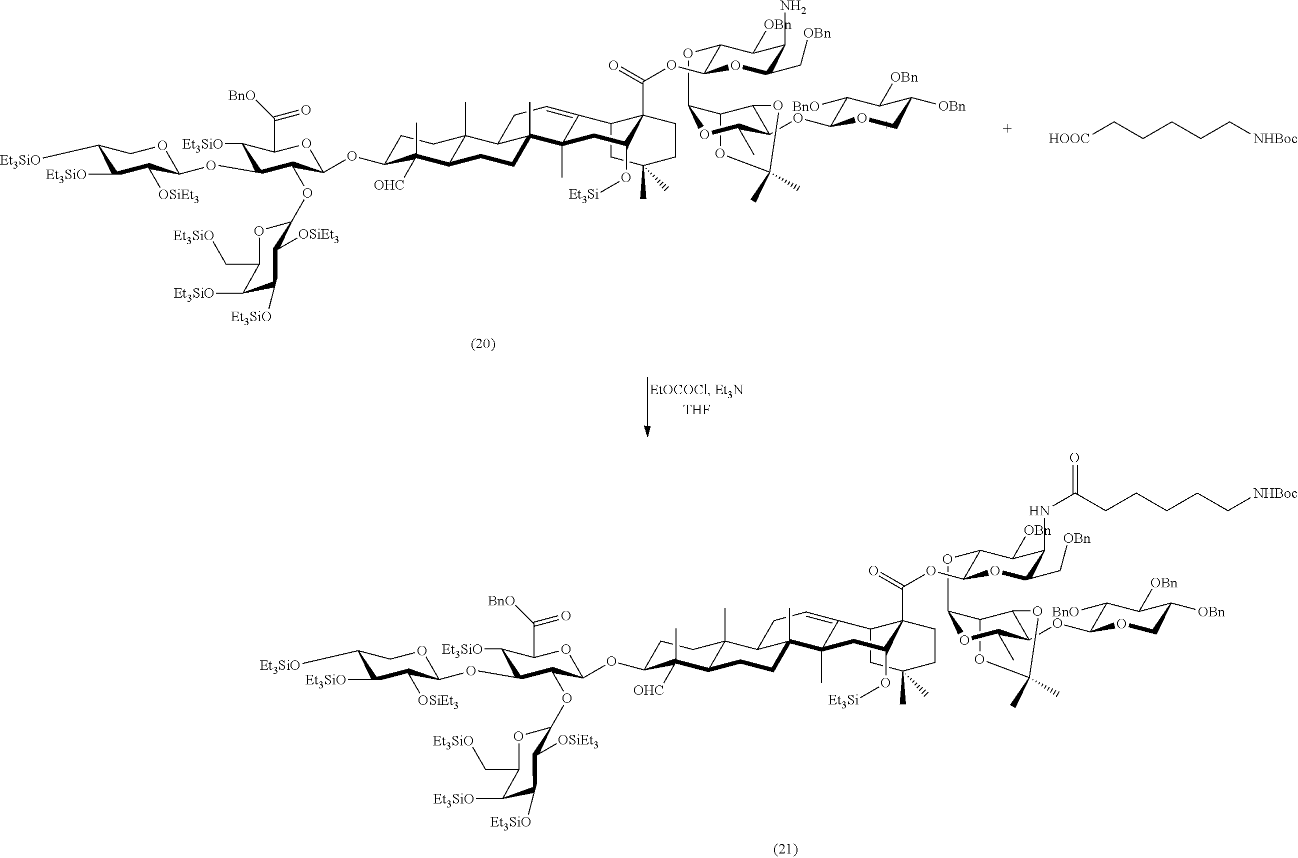

In a 10-mL pear-shaped Schlenk flask, commercially available 6-(Boc-amino)hexanoic acid (HO₂C(CH₂)₅NHBoc) (90 mg, 0.382 mmol, 11.5 equiv.) is azeotropically dried and then dissolved in THE (5 mL), then Et₃N (0.45 mL, 2.99 mmol, 90 equiv.) is added. To this clear, colorless solution at 0° C., EtOCOCl (35 μL, 0.33 mol, 9.0 equiv.) is injected via gas-tight syringe. The turbid white mixture is stirred for 2 h at 0° C. In another 25 mL Schlenk flask, the prosapogenin-linear trisaccharide saponin amine 20 (100 mg, 0.03327 mmol, 1.0 equiv.) is azeotropically dried, and the activated acid is then cannula transferred to it at ice bath condition, stirred 0° C.-rt for 2 h. The reaction is quenched by adding water, diluted with DCM and extracted with NaHCO₃. Organic layer was concentrated and purified by silica gel chromatography (toluene/EtOAc, 20:1 to 11:1) to afford the aminoacyl, branched trisaccharide-containing saponin 21 (90 mg, 87%) as a white glassy solid. R_{f}=0.52 (toluene/ethyl acetate 4:1)¹H NMR (400 MHz, Benzene-d₆) δ 9.73 (s, 1H), 7.52-7.48 (m, 5H), 7.36 (t, J=7.0 Hz, 5H), 7.27 (d, J=2.1 Hz, 1H), 7.25 (t, J=1.7 Hz, 2H), 7.23 (d, J=1.7 Hz, 2H), 7.21 (s, 2H), 7.20-7.19 (m, 18H), 7.09 (tdd, J=9.4, 6.5, 4.7 Hz, 7H), 5.70-5.65 (m, 2H), 5.58 (d, J=3.5 Hz, 1H), 5.29 (d, J=10.2 Hz, 1H), 5.21 (d, J=6.9 Hz, 1H), 5.18 (d, J=2.0 Hz, 1H), 5.13 (d, J=11.3 Hz, 1H), 5.04 (s, 1H), 5.00 (d, J=5.6 Hz, 1H), 4.94 (s, OH), 4.91 (s, 2H), 4.87 (s, 1H), 4.85 (d, J=3.2 Hz, 2H), 4.82 (d, J=4.3 Hz, 1H), 4.76 (d, J=7.2 Hz, 1H), 4.64 (d, J=6.9 Hz, 1H), 4.55-4.46 (m, 5H), 4.43 (d, J=5.0 Hz, 1H), 4.41 (d, J=1.8 Hz, 2H), 4.39-4.35 (m, 1H), 4.33 (s, OH), 4.23 (dt, J=5.3, 3.3 Hz, 3H), 4.18 (d, J=9.3 Hz, 1H), 4.12 (ddt, J=9.5, 7.6, 3.9 Hz, 2H), 4.07-4.01 (m, 1H), 3.97 (qd, J=7.6, 6.4, 3.8 Hz, 2H), 3.89 (dd, J=11.5, 5.3 Hz, 1H), 2.89 (q, J=6.7 Hz, 2H), 2.52 (t, J=13.5 Hz, 1H), 2.22 (d, J=6.5 Hz, 3H), 2.13-2.06 (m, 1H), 1.98 (td, J=11.3, 10.8, 4.4 Hz, 1H), 1.88 (d, J=9.6 Hz, 1H), 1.60 (s, 3H), 1.57 (d, J=6.2 Hz, 3H), 1.53 (d, J=3.6 Hz, 6H), 1.48 (s, 12H), 1.35 (s, 6H), 1.29 (d, J=2.3 Hz, 6H), 1.27 (s, 4H), 1.25 (s, 3H), 1.23 (s, 1H), 1.21 (d, J=2.1 Hz, 8H), 1.20-1.18 (m, 14H), 1.17 (d, J=1.4 Hz, 11H), 1.15 (s, 6H), 1.12-1.01 (m, 45H), 1.01-0.85 (m, 28H), 0.74 (qt, J=7.3, 3.1 Hz, 22H). ¹³C NMR (101 MHz, C₆D₆) δ 209.21, 175.11, 172.40, 168.77, 155.79, 143.69, 139.71, 139.54, 139.11, 138.49, 138.37, 135.89, 130.76, 128.93, 128.65, 128.25, 128.01, 127.77, 122.66, 109.78, 103.16, 102.67, 101.72, 101.56, 98.39, 94.87, 84.26, 83.49, 82.70, 80.02, 79.76, 79.41, 79.29, 79.00, 78.55, 78.17, 77.13, 76.94, 76.76, 76.46, 75.54, 74.92, 74.76, 73.76, 73.51, 73.29, 73.14, 73.07, 72.44, 71.79, 71.75, 68.51, 68.14, 68.01, 67.04, 66.14, 64.17, 61.60, 54.75, 49.48, 49.06, 47.20, 46.82, 46.49, 41.95, 41.25, 40.57, 40.40, 39.18, 38.18, 37.56, 36.23, 36.09, 35.74, 35.34, 33.22, 33.04, 32.85, 32.32, 31.55, 30.79, 30.52, 30.19, 30.12, 30.03, 29.81, 29.28, 28.60, 27.77, 27.59, 27.24, 26.83, 26.30, 25.96, 25.51, 24.99, 24.58, 24.13, 23.73, 23.33, 23.11, 22.86, 20.71, 18.34, 17.45, 16.02, 14.36, 11.89, 11.15, 7.93, 7.77, 7.62, 7.57, 7.46, 7.39, 7.19, 6.43, 6.22, 6.10, 6.00, 5.94, 5.83, 5.77, 5.36, 4.92.

### (VII) Global Deprotection of Protected Branched Trisaccharide-Containing Aminoacylated Saponin Precursor 21 to Synthesize Aminoacyl Saponin 22, by Hydrogenolysis and Acid Hydrolysis (AS-III-045):

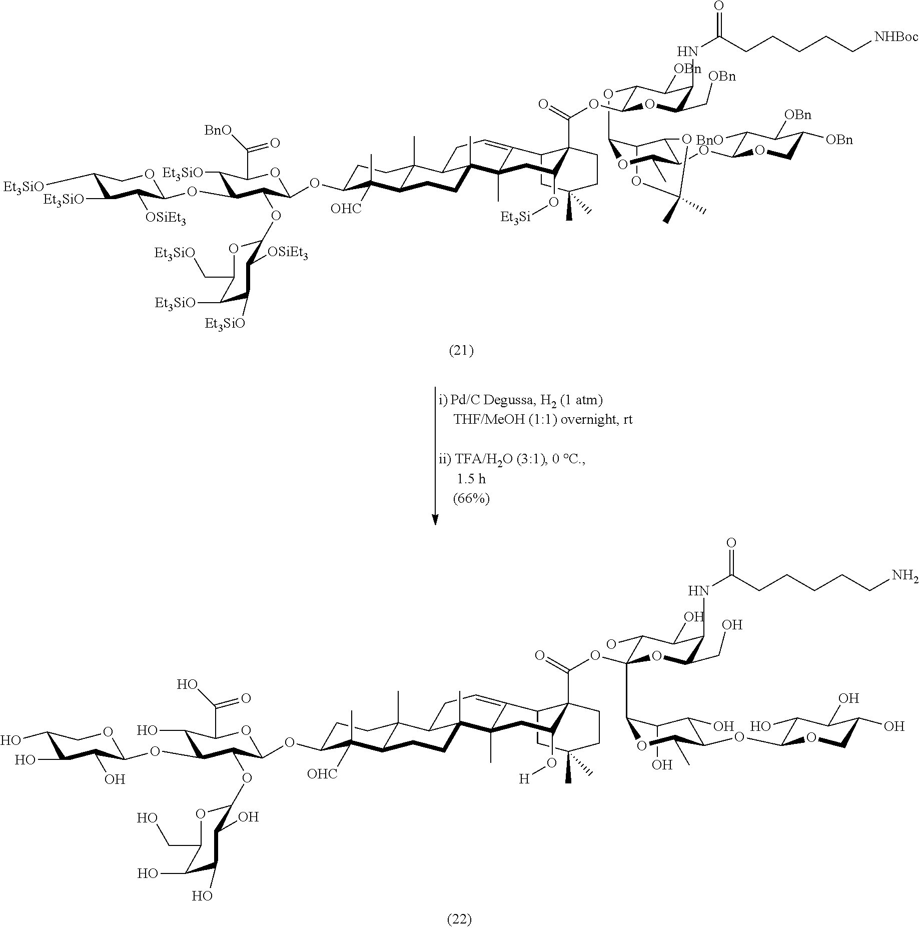

In a 25-mL round-bottomed flask, fully protected, azeotropically dried branched trisaccharide-containing saponin 21 (22 mg, 6.83 μmol, 1.0 equiv.) is dissolved in THF/MeOH (1:1) (10 mL), then 10% (dry basis) Pd/C, wet (50% water), Degussa type E101 NE/W (8 mg, 75 μmol, 11 equiv.) is added. The reaction mixture is stirred at rt for overnight under H₂ atmosphere (1 atm) using ballon. The suspension is filtered through a 0.45 μm nylon syringe filter (Fischer Brand) and the palladium is washed thoroughly with MeOH (3×15 mL) and the clear filtrate is concentrated. Successful debenzylation is assessed by the disappearance of aromatic resonances by ¹H-NMR in methanol-d₄. In a 25-mL round-bottomed flask, the resulting crude mixture of partially desilylated products is dissolved in a precooled (0° C.) solution of TFA/water (4:1) (5 mL). The reaction mixture is stirred for 1.5 h at 0° C. and then concentrated carefully under high vacuum at 0° C. and dissolved in ACN/Water (1:1) and lyophilized to give a white solid residue. This lyophilized solid is dissolved in a solution of water/MeCN (4:1) and purified by RP-HPLC PREP column using a linear gradient of 20→100% CH3CN in water (0.05 vol % TFA) in 30 min. The fraction containing the major, single peak with t_{R}=5.85 min is collected and lyophilized to dryness to afford the fully deprotected, free amine-containing saponin 22 (6.79 mg, 66%) as a white solid. The purity of the product was checked by C18 analytical column using a linear gradient of 5→100% CH₃CN in water (0.05 vol % TFA) in 30 min the same gradient as shown below. The pure product appeared at t_{R}=14.11 min, λₘₐₓ=193.12 nm.

### (B) Synthesis of NHS Ester of Diazirine Containing Photo-Linker, Compound 30:

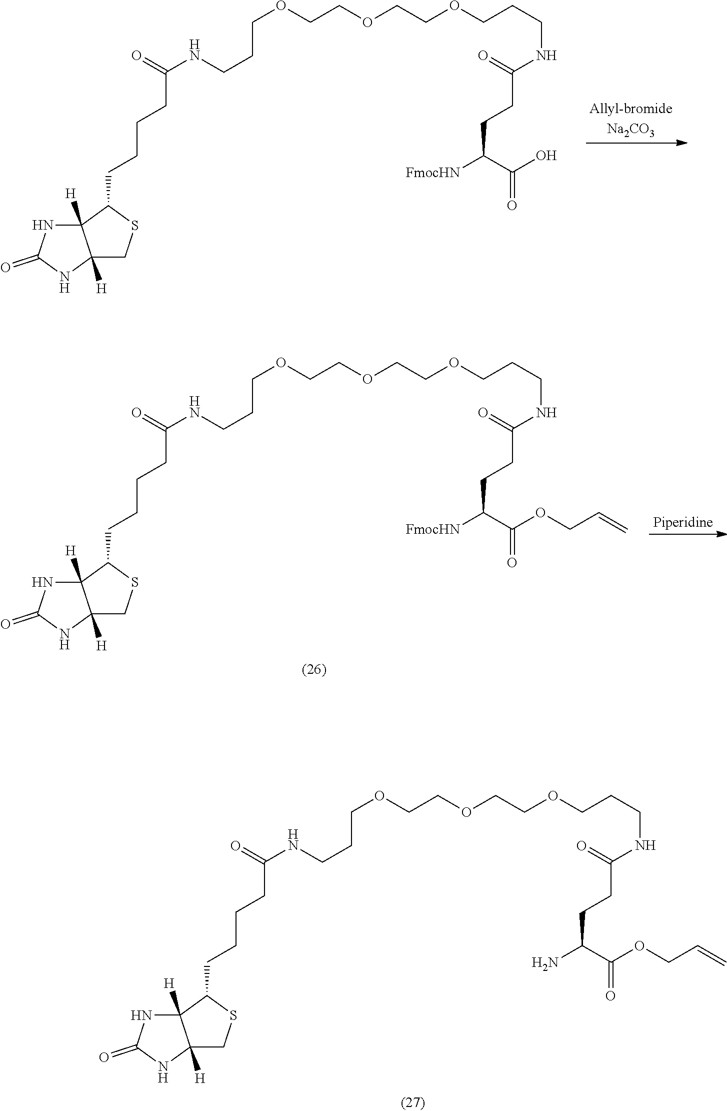

(a) Synthesis of H₂N-Glu(Biot)-OAII, 27 from commercially available Fmoc-Glu(Biot)-OH (AS-II-046):

In a 10 mL round bottom flask, commercially available Fmoc-Glu(Biot)-OH (150 mg, 0.1879 mmol, 1 eqv.) was dissolved in 3 mL dry DMF and added solid Na₂CO₃ (23 mg, 0.2255 mmol, 1.2 eqv.) which resulted a fine suspension. To the suspension, allylbromide (20 μL, 0.2255 mmol, 1.2 eqv.) was added using a gas tight syringe and stirred at 40° C. for overnight. The mixture was concentrated using high vacuum pump at 30° C. and then diluted with DCM (80 mL) and washed with water (50 mL×2). The organic layer was concentrated; obtained 175 mg of the crude which was used in the next step without further purification. Again, the crude (175 mg) was dissolved in 4 mL DMF, and piperidine (180 μL, 10 eqv.) was added. After 1 h, complete disappearance of the starting material observed in TLC. The crude was concentrated and purified by silica gel chromatography (using the solvent gradient pure DCM to DCM/MeOH 8.5:1.5 with 1% triethylamine which afford the deprotected H₂N-Glu(Biot)-OAII 27 (105 mg, 90%) as a transparent glassy solid.

### (b) Synthesis of Diazirine Containing Photo-Linker, 28 Via Coupling Between Dazirine-000H and H₂N-Glu(Biot)-OAII 27 (AS-III-003):

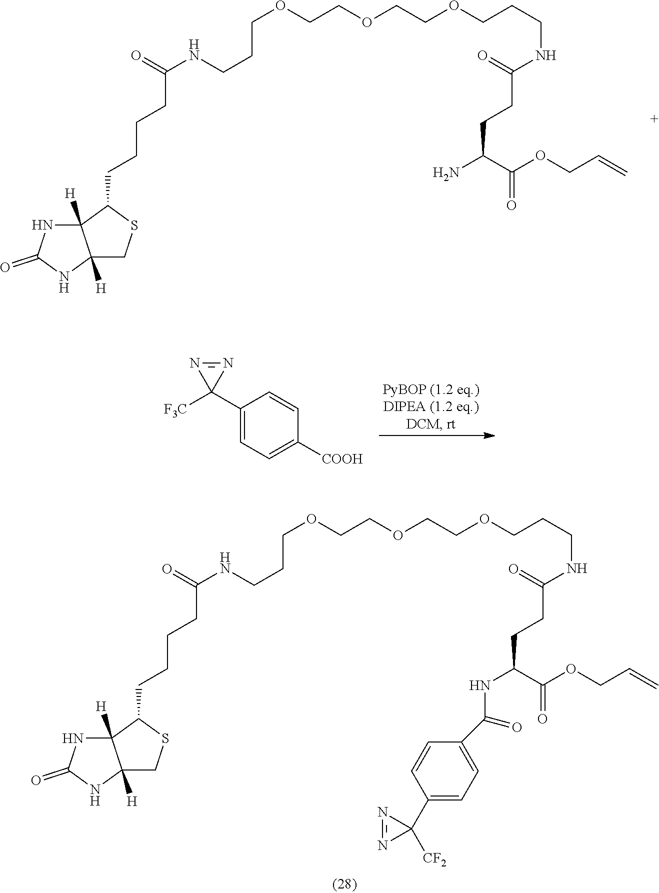

In a 10 mL round bottom flask, H₂N-Glu(Biot)-OAII, 27 (95 mg, 0.154 mmol, 1 eqv.) was dissolved in 3.2 mL dry DCM. And then added DIPEA (35 μL, 0.201 mmol, 1.3 eqv.), Dazirine-COOH (55 mg, 0.238 mmol, 1.5 eqv.) and PyBOP (110 mg, 0.211 mmol, 1.37 eqv.) giving a clear solution. The mixture was then stirred at rt for 2-3 h giving a bright new spot in the TLC. The mixture was concentrated by N₂ flow and purified by silica gel chromatography using the solvent gradient DCM/MeOH 9.5:0.5 to 9:1 with 1% TEA affording the desire product DAz-Glu(Biot)-OAII, 28 (120 mg, 94%) as glassy solid.

### (c) Synthesis of DAz-Glu(Biot)-OH, 29 Via Deallylation of 28 (AS-III-004):

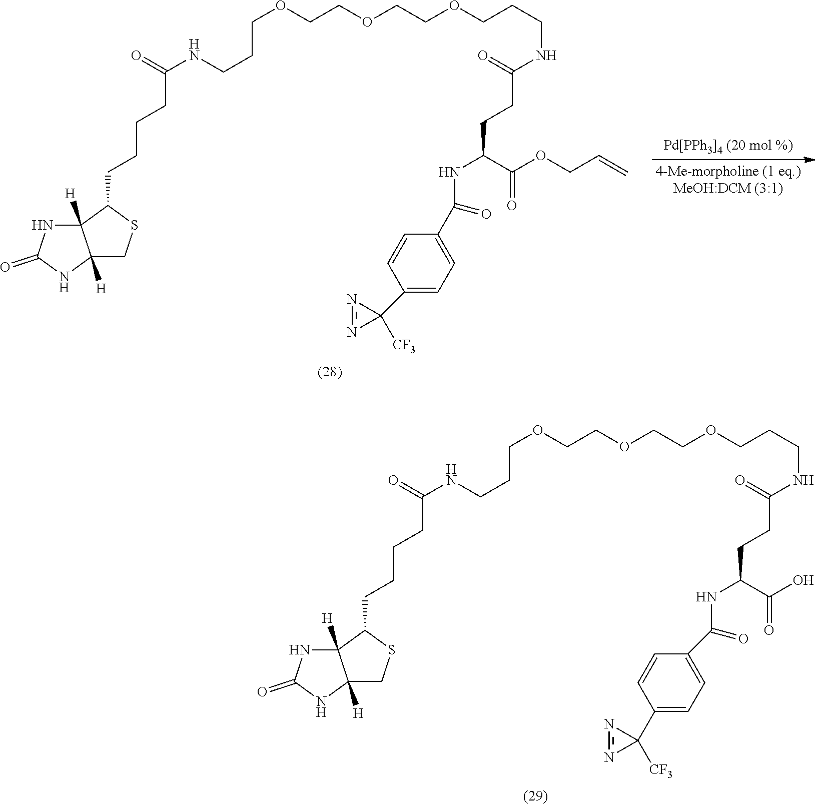 [SMILES: C=CCOC(=O)[C@H](CCC(=O)NCCCOCCOCCOCCCNC(=O)CCCC[C@@H]1SC[C@@H]2NC(=O)N[C@@H]21)NC(=O)c1ccc(C2(C(F)(F)F)N=N2)cc1 ; O=C(CCCC[C@@H]1SC[C@@H]2NC(=O)N[C@@H]21)NCCCOCCOCCOCCCNC(=O)CC[C@H](NC(=O)c1ccc(C2(C(F)(F)F)N=N2)cc1)C(=O)O]

In a 25 mL modified Schlenk flask, azeotropically dried DAz-Glu(Biot)-OAII, 28 (150 mg, 0.181 mmol, 1 eqv.) is dissolved in MeOH/DCM (5 mL, 4:1) and added 4-methylmorpholine (19 μL, 0.181 mmol, 1 eqv.). The mixture is degassed by performing three freeze-thaw-pump cycles. After the degas, (triphenylphosphine)palladium (0) (41 mg, 0.036 mmol, 0.2 eqv.) was added and the mixture was degassed again by performing the same freeze-thaw cycle×3. The mixture is stirred at rt overnight. The crude was concentrated and dissolved in MeCN/H₂O (1:1, 5 mL), purified by RP C18 EcoFlexFlash 4 g column chromatography (Buchi) using the solvent gradient 5-100% Acetonitrile (0.05% TFA) in 25 min which affords the desire product DAz-Glu(Biot)-OH, 29 (118 mg, 83%) as glassy solid. ¹H NMR (400 MHz, Methanol-d₄) δ 8.05-7.96 (m, 2H, H-Ph), 7.39 (d, J=8.2 Hz, 2H, H-Ph), 4.61 (dd, J=9.3, 4.5 Hz, 1H, H-Biotin), 4.52 (dd, J=7.9, 4.8 Hz, 1H, H-αCH (Glu)), 4.33 (dd, J=7.9, 4.4 Hz, 1H, H-Biotin), 4.13 (q, J=7.1 Hz, OH), 3.68-3.63 (m, 4H), 3.60 (ddd, J=5.9, 4.4, 2.8 Hz, 4H), 3.53 (q, J=6.3 Hz, 3H), 3.32-3.20 (m, 5H), 2.95 (dd, J=12.7, 5.0 Hz, 1H), 2.73 (d, J=12.7 Hz, 1H), 2.47-2.27 (m, 3H), 2.28-2.07 (m, 3H), 2.04 (s, OH), 1.84-1.55 (m, 7H), 1.46 (p, J=7.3 Hz, 2H), 1.37-1.23 (m, 1H).

### (d) Synthesis of NHS Ester of DAz-Glu(Biot)-OH, 30 (AS-III-023):

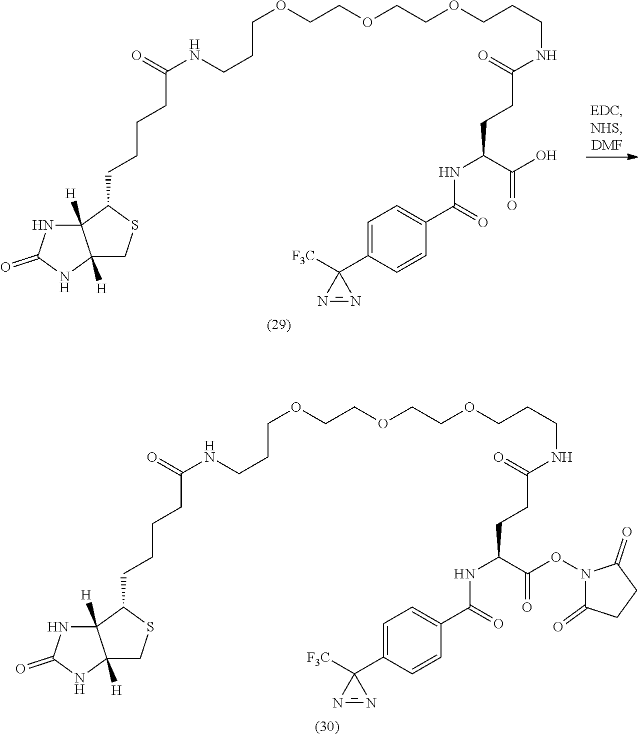 [SMILES: O=C(CCCC[C@@H]1SC[C@@H]2NC(=O)N[C@@H]21)NCCCOCCOCCOCCCNC(=O)CC[C@H](NC(=O)c1ccc(C2(C(F)(F)F)N=N2)cc1)C(=O)O ; O=C(CCCC[C@@H]1SC[C@@H]2NC(=O)N[C@@H]21)NCCCOCCOCCOCCCNC(=O)CC[C@H](NC(=O)c1ccc(C2(C(F)(F)F)N=N2)cc1)C(=O)ON1C(=O)CCC1=O]

In a 25 mL round bottom flask, DAz-Glu(Biot)-OH, 29 (13 mg, 0.0165 mmol, 1 eqv.), EDC (30 mg, 0.165 mmol, 10 eqv.), NHS (18.8 mg, 0.165 mmol, 10 eqv.) are dissolved in 1.5 mL DMF in ice bath condition. The mixture is stirred 0° C.-rt for 2 h under argon atmosphere. After 2 h, another 15 mg of EDC and 15 mg of NHS was added and stirred at rt for another 1 h. The mixture was purified in Buchi by RP C18 PREP mode using a linear gradient 5-100% ACN in water with 0.05% TFA over 25 min (at flow rate 20 mL/min) afford the desired NHS activated ester i.e. DAz-Glu(Biot)-NHS, 30 (7.29 mg, 49%). It is to be noted that the purified product also contains a little amount of the hydrolysed starting material i.e DAz-Glu(Biot)-OH, 29. After the purification purity of the product was checked in analytical C18 column using a linear gradient 5-100% ACN in water with 0.05% TFA over 30 min. The pure product appeared at t_{R}=18.59 min, λ₁ₘₐₓ=198.52 nm. λ₂ₘₐₓ=234.52 along with hydrolysed product appeared at t_{R}=17 min. [Caution: Care must be taken while dealing with the NHS activated ester of DAz-Glu(Biot)-OH since the compound is extremely labile. During the synthesis it was found that the activated ester decomposed very easily. To avoid such incidences, it is recommended to use an ice bath condition during purification and afterwards. This NHS ester should be used as soon as possible for the next step of conjugation thus avoiding long term storage resulting degradation.]

### Chemical Synthesis of BTS-25-BP

### (C) Synthesis of Benzophenone Containing Photo-Linker, Compound 24:

(i) Synthesis of NHS Ester of Benzophenone-4,4′-dicarboxylic Acid (BP-NHS), 23 (AS-II-012):

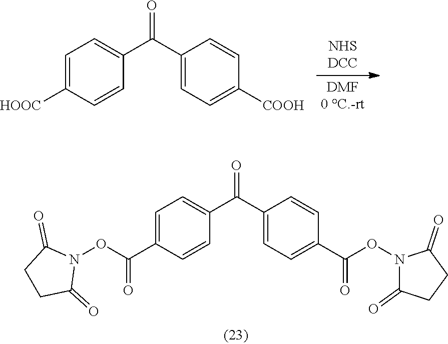 [SMILES: O=C(O)c1ccc(C(=O)c2ccc(C(=O)O)cc2)cc1 ; O=C(ON1C(=O)CCC1=O)c1ccc(C(=O)c2ccc(C(=O)ON3C(=O)CCC3=O)cc2)cc1]

In a 50 mL round bottom flask, Benzophenone-4,4′-dicarboxylic Acid (200 mg, 0.74 mmol, 1 eqv.) was dissolved in 6 mL dry DMF. To it N-hydroxysuccinamide (280 mg, 2.4 mmol, 3.2 eqv.) and DCC (550 mg, 3.6 mmol, 3.6 eqv.) was added in ice bath condition and then stirred 0° C.-rt for 30 min. The crude reaction mixture was filtered through celite. The crude then washed with Sodium bicarbonate (30 mL) and extracted with ethylacetate (100 mL×3). The organic layer concentrated and purified by silica gel column chromatography using the solvent system hexane/ethylacetate (1:1.25) which afford the NHS ester of Benzophenone-4,4′-dicarboxylic Acid, 23 as white solid (340 mg, 99%).

(ii) Conjugation of BP-NHS, 23 with the Commercially Available Biotin-PEG5-NH₂ to Synthesize Biot-PEG5-BP-NHS, 24 (AS-II-023):

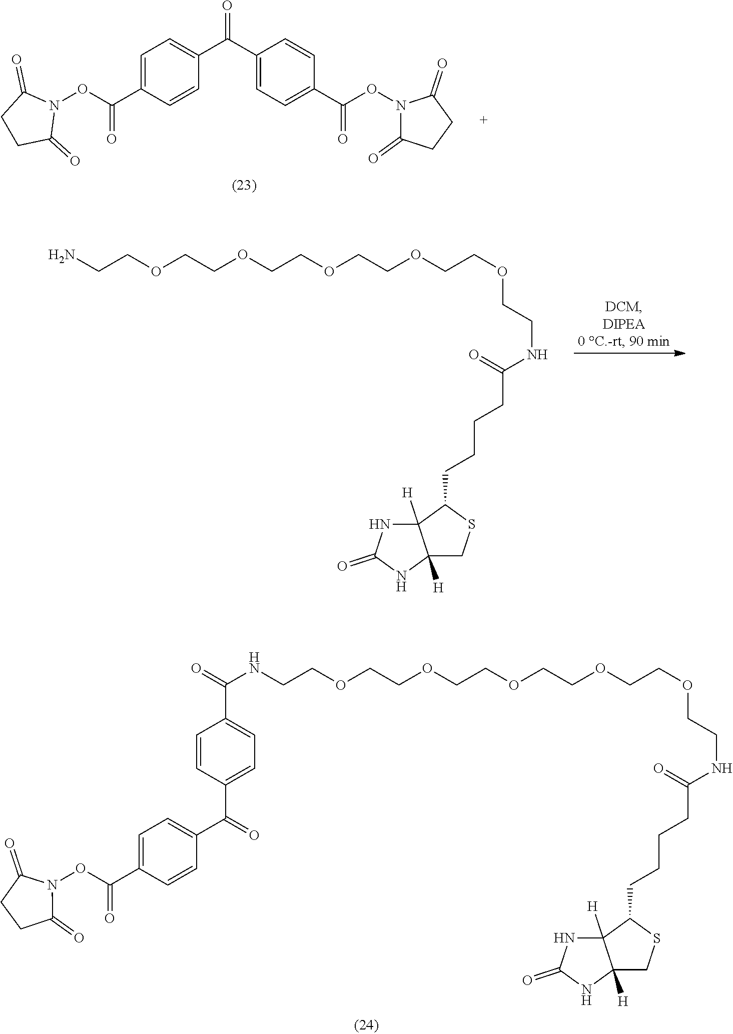 [SMILES: O=C(ON1C(=O)CCC1=O)c1ccc(C(=O)c2ccc(C(=O)ON3C(=O)CCC3=O)cc2)cc1 ; NCCOCCOCCOCCOCCOCCNC(=O)CCCC[C@@H]1SC[C@@H]2NC(=O)NC21 ; O=C(CCCC[C@@H]1SC[C@@H]2NC(=O)NC21)NCCOCCOCCOCCOCCOCCNC(=O)c1ccc(C(=O)c2ccc(C(=O)ON3C(=O)CCC3=O)cc2)cc1]

In a 5 mL small vial, Biotin-PEG5-NH₂ (17 mg, 0.033 mmol, 1 eqv.) was dissolved in 1 mL DMF. To it DIPEA (16 μL, 0.097 mmol, 2.5 eqv.) was added and stirred at rt for 10 min. In another vial, BP-NHS, 23 (110 mg, 0.23 mmol, 7.18 eqv.) dissolved in 2 mL dry DMF which was then transferred to the reaction mixture slowly and stirred at rt for 90 min. The reaction was quenched by adding 0.5 mL of acetonitrile with 0.05% TFA. Purification was done in RP C18 PREP column using a linear gradient 5-100% ACN in water with 0.05% TFA over 30 min. The fraction containing the desired, single peak is collected and lyophilized to dryness to afford the Biot-PEG5-BP-NHS, 24 (13 mg, 46%) as a sticky glassy solid. The purity of the product with was checked by C18 analytical column using the same gradient as shown below. The pure product appeared at t_{R}=16.07 min, λ₁ₘₐₓ=198.52 nm. λ₂ₘₐₓ=262.52. ¹H NMR (400 MHz, Chloroform-d) δ 8.27 (d, J=8.0 Hz, 2H), 8.02 (d, J=7.7 Hz, 2H), 7.87 (dd, J=24.2, 7.6 Hz, 4H), 6.84 (s, 1H), 6.31 (s, 1H), 5.72 (s, 1H), 5.30 (s, OH), 4.55 (s, 1H), 4.36 (s, 1H), 4.28-4.15 (m, OH), 3.61 (dd, J=41.0, 19.6 Hz, 23H), 3.42 (s, 2H), 3.16 (s, 1H), 2.94 (s, 4H), 2.74 (d, J=12.4 Hz, 1H), 2.25 (s, 18H), 0.91 (dt, J=9.3, 7.0 Hz, 1H).

### D) Chemical Assembly of Biotin-Benzophenone Containing Saponin, Compound BTS-25-BP:

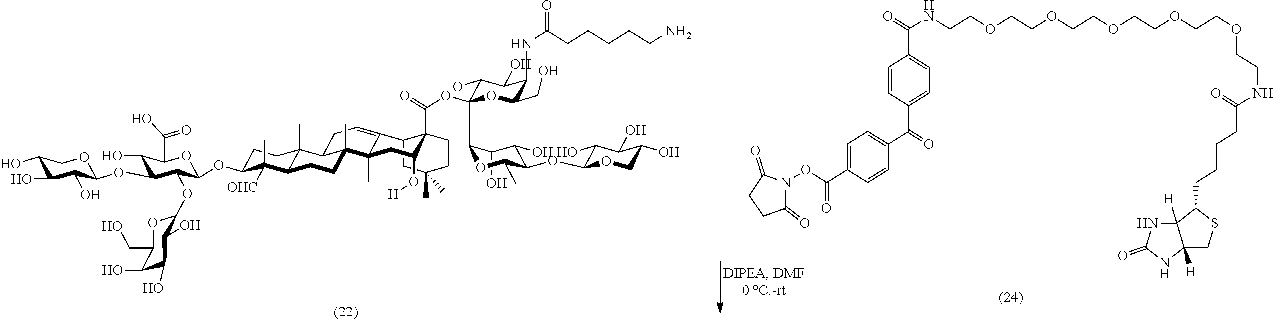

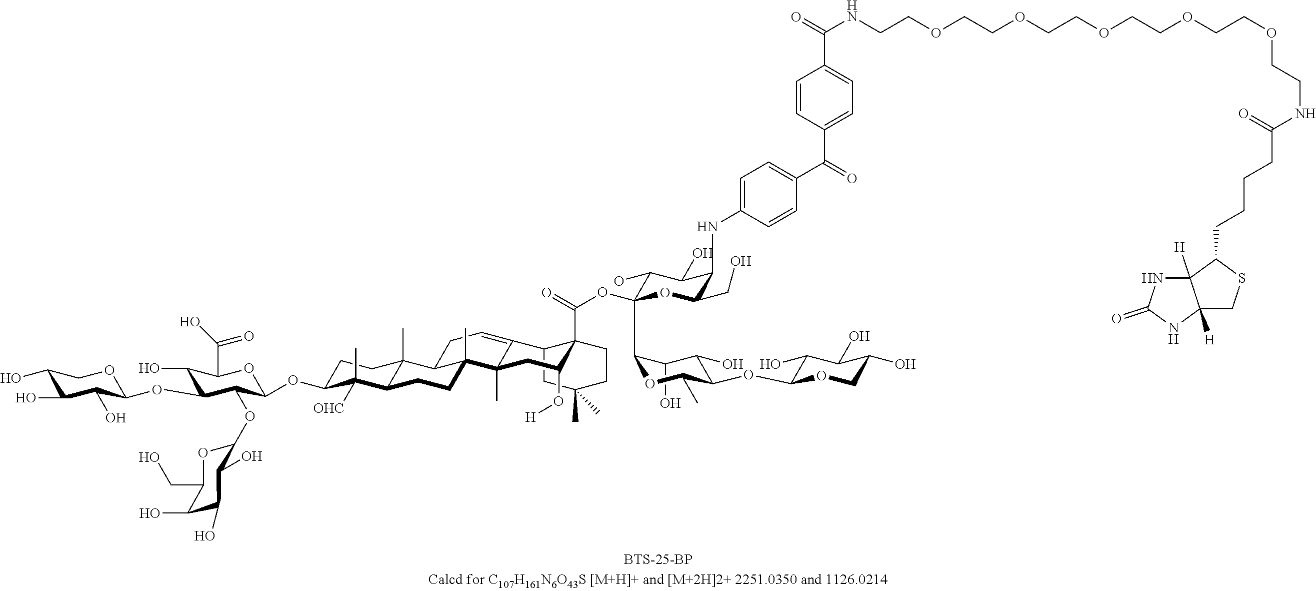

In a small vial, aminoacyl Saponin 22 (3.38 mg, 2.24 μmol, 1 eqv.) was dissolved in 0.7 mL dry DMF and cooled to 0° C. And then DIPEA (1 μL, 5.6 μmol, 2.5 eqv.) was added to it. In another vial, the NHS activated ester 24 (2.29 mg, 2.7 μmol, 1.2 eqv.) was dissolved in 1 mL dry DMF and then transferred to the saponin mixture via a gas tight glass syringe and stirred at 0° C.-rt for 2-3 h. The reaction was quenched by adding 1 mL of cold water in ice bath condition; purified by RP-HPLC PREP column using a linear gradient of 20→70% CH₃CN in water (0.05 vol % TFA) in 30 min. The fraction containing the major, single peak with t_{R}=15.11 min is collected and lyophilized to dryness to afford the benzophenone containing saponin photo-affinity probe, BTS-25-BP (1.99 mg, 59%) as a white solid. The purity of the product was checked by C18 analytical column using a linear gradient of 5→100% CH₃CN in water (0.05 vol % TFA) in 30 min. The pure product appeared at t_{R}=16.07 min, λ₁ₘₐₓ=193.52 nm. λ₂ₘₐₓ=267.52. 1 H NMR (400 MHz,) δ 9.33 (s, 1H), 7.95-7.79 (m, 8H), 5.32-5.22 (m, 2H), 5.13 (d, J=1.5 Hz, 1H), 4.66 (d, J=7.8 Hz, 1H), 4.56 (d, J=7.8 Hz, 1H), 4.37-4.29 (m, 2H), 4.26 (dd, J=8.0, 4.5 Hz, 2H), 3.93-3.78 (m, 4H), 3.77-3.68 (m, 3H), 3.68-3.40 (m, 21H), 3.39-3.26 (m, 5H), 3.26-3.09 (m, 6H), 2.83 (p, J=7.1, 6.1 Hz, 2H), 2.70-2.57 (m, 1H), 2.30 (tt, J=15.0, 7.3 Hz, 2H), 2.11 (q, J=6.3, 5.4 Hz, 3H), 1.91-1.43 (m, 11H), 1.42-1.08 (m, 12H), 1.05 (s, 3H), 0.88 (s, 2H), 0.83 (s, 3H), 0.77 (s, 3H), 0.63 (s, 3H). MS (ESI+) m/z: Calcd for [C₁₀₇H₁₆₁N₆O₄₃S]⁺ [M+H]⁺ 2251.0350, found: 2249.65.

### III. In Vivo Immunological Evaluation

### In Vivo Experiment 1: Toxicity Assessment of BTS-31-DAz and BTS-25-BP Vs QS-21

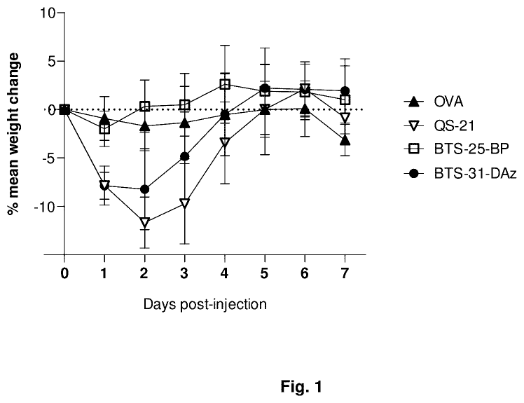

Mouse weight loss was monitored after each injection (1^{st}, 2^{nd} and 3^{rd} (booster dose)). QS21 showed highest toxicity (about 12%) whereas BTS-31-DAz and BTS-25-BP showed only about 7-8% and about 2% respectively. See FIG. 1.

### In Vivo Experiment 2: Adjuvant Activity of BTS-31-DAz Vs QS-21

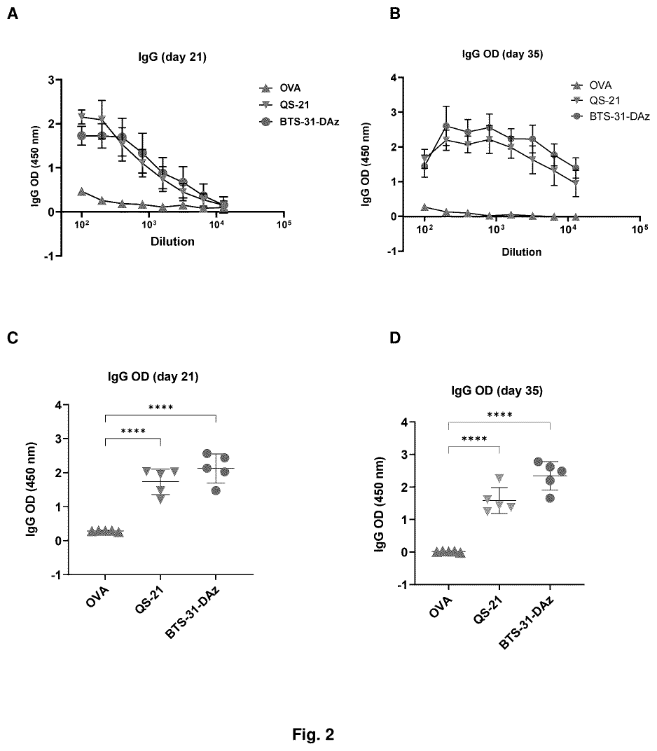

We evaluated adjuvant activity in a mouse vaccination model involving coadministration with ovalbumin (OVA) as an immunogen. Groups of five mice (female C57BL/6, 6-8 weeks of age) were immunized with endotoxin-free OVA (EndoFit™ Ovalbumin; Invitrogen) (10 μg/mice) and the saponin of interest (20 μg/mouse) in phosphate-buffered saline (PBS, 100 μL) via subcutaneous injections on days 0 and 14, followed by a booster dose on day 28. As positive control mice were immunized with natural QS-21, and as negative control with OVA antigen only. Mouse blood was collected one week before (day 21) and one week after the booster (third) immunization, at the time of sacrifice (day 35). The presence of antibodies against OVA in sera was detected by ELISA at both time points (FIGS. 2(a, b)).

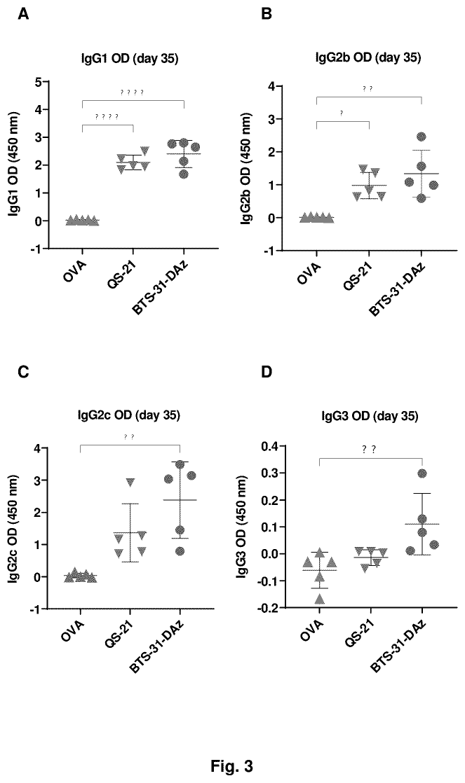

On day 21, BTS-31-DAz elicited significantly higher anti-OVA IgG levels compared to the OVA-alone (no adjuvant) negative control (FIG. 2a). Compared to the positive control QS-21, BTS-31-DAz showed slightly higher antibody response. The above trend was maintained on day 35 as well (at the time of sacrifice) with higher IgG response elicited by BTS-31-DAz (FIG. 2b) over the QS-21 group. In terms of IgG titers, BTS-31-DAz induced significantly higher IgG antibodies than QS-21 (p<0.05; p=0.0209) as shown in FIG. 2 (c-d). Additionally, we assessed IgG subtyping such as IgG1, IgG2 and IgG3 subclasses of the anti-OVA antibodies (day 35) for the above saponins and compared to that of total IgG (FIG. 3). In each case, the antibody response for the BTS-31-DAz over OVA alone was significantly higher. When compared with the positive control, in case of IgG1, the antibody response elicited by BTS-31-DAz over QS21 was higher. In case of IgG2 isotypes, BTS-31-DAz also showed significantly higher antibody levels for both IgG2b (FIGS. 3b) and IgG2c (FIGS. 3c) subclass than QS21. Moreover, in case of IgG3, although the antibody response was lower, BTS-31-DAz showed higher antibody response than QS21.

Since IgG2a (IgG2c in C57BL/6 mice) production is associated to Th1 immunity and IgG1 production is associated with Th2 immunity, just like QS21, the higher levels of these subclasses induced by the BTS-31-DAz points to a similar, mixed Th1/Th2 response.

Together these data shows that the compounds of formula (I) such as BTS-31-DAz and BTS-25-BP are superior to the original QS21 in terms of potency, toxicity or both.

### LIST OF REFERENCES

Ahmad, T. A., et al. Trials in Vaccinology 2016, Volume 5, Pages 71-83. https://doi.org/10.1016/j.trivac.2016.04.003.
Bergmann-Leitner, E.; Leitner, W. Adjuvants in the Driver's Seat: How Magnitude, Type, Fine Specificity and Longevity of Immune Responses Are Driven by Distinct Classes of Immune Potentiators. Vaccines 2014, 2 (2), 252-296. https://doi.org/10.3390/vaccines2020252.
Buskas, T.; Ingale, S.; Boons, G.-J. Towards a Fully Synthetic Carbohydrate-Based Anticancer Vaccine: Synthesis and Immunological Evaluation of a Lipidated Glycopeptide Containing the Tumor-Associated Tn Antigen. Angew. Chemie Int. Ed. 2005, 44 (37), 5985-5988. https://doi.org/10.1002/anie.200501818.
Chea, E. K.; Fernández-Tejada, A.; Damani, P.; Adams, M. M.; Gardner, J. R.; Livingston, P. O.; Ragupathi, G.; Gin, D. Y. Synthesis and Preclinical Evaluation of QS-21 Variants Leading to Simplified Vaccine Adjuvants and Mechanistic Probes. J. Am. Chem. Soc. 2012, 134, 13448-13457.
Doe, B.; Steimer, K. S.; Walker, C. M.; Induction of HIV-1 envelope (gp120)-specific cytotoxic T lymphocyte responses in mice by recombinant CHO cell-derived gp120 is enhanced by enzymatic removal of N-linked glycans. Eur J Immunol. 1994 October; 24(10):2369-76. https:/doi.org/10.1002/eji.1830241017.
Dönnes, P.; Kohlbacher, O. Integrated modeling of the major events in the MHC class I antigen processing pathway. Protein Sci. 2005 August; 14(8):2132-40. https://doi.org/10.1110/ps.051352405.
Doytchinova, I. A.; Guan, P.; Flower, D. R. EpiJen: a server for multistep T cell epitope prediction. BMC Bioinformatics 2006 Mar. 13; 7:131. https://doi.org/10.1186/1471-2105-7-131.
Erickson, A. L.; Houghton, M.; Choo, Q. L.; Weiner, A. J., Ralston, R.; Muchmore, E.; Walker, C. M.; Hepatitis C virus-specific CTL responses in the liver of chimpanzees with acute and chronic hepatitis C. J Immunol. 1993 Oct. 15; 151(8):4189-99.
Fernández-Tejada, A.; Chea, E. K.; George, C.; Pillarsetty, N.; Gardner, J. R.; Livingston, P. O.; Ragupathi, G.; Lewis, J. S.; Tan, D. S.; Gin, D. Y. Development of a Minimal Saponin Vaccine Adjuvant Based on QS-21. Nat. Chem. 2014, 6 (7), 635-643. https://doi.org/10.1038/nchem.1963
Fernández-Tejada A. Design, Synthesis and Evaluation of Optimized Saponin Variants Derived From the Vaccine Adjuvant QS-21. Pure Appl Chem (2017) 89:1359-78. doi: 10.1515/pac-2016-1213
R. Fuentes, L. Aguinagalde, C. Pifferi, A. Plata, N. Sacristán, D. Castellana, J. Anguita and A. Fernández-Tejada, Front. Immunol., 2022, 13, 1-11.
Ghirardello, M.; Ruiz-de-Angulo, A.; Sacristan, N.; Barriales, D.; Jiménez-Barbero, J.; Poveda, A.; Corzana, F.; Anguita, J.; Fernández-Tejada, A. Exploiting Structure-activity Relationships of QS-21 in the Design and Synthesis of Streamlined Saponin Vaccine Adjuvants. Chem. Commun. 2020, 56 (5), 719-722. https://doi.org/10.1039/C9CC07781B.
Kozoriz K, Shkel O, Hong K T, Kim D H, Kim Y K, Lee J S. Multifunctional Photo-Cross-Linking Probes: From Target Protein Searching to Imaging Applications. Acc. Chem. Res. 2023 Jan. 3; 56(1):25-36. https://doi.org/10.1021/acs.accounts.2c00505
Pifferi, C.; Fuentes, R.; Fernández-Tejada, A. Natural and Synthetic Carbohydrate-Based Vaccine Adjuvants and Their Mechanisms of Action. Nat. Rev. Chem. 2021, 25 (4), 3-7. https://doi.org/10.1038/s41570-020-00244-3.
Reche, P. A.; Glutting, J. P.; Zhang, H.; Reinherz, E. L. Enhancement to the RANKPEP resource for the prediction of peptide binding to MHC molecules using profiles. Immunogenetics 2004 September; 56(6):405-19. https://doi.org/10.1007/s00251-004-0709-7.
Reed, S. G.; Orr, M. T.; Fox, C. B. Key Roles of Adjuvants in Modern Vaccines. Nat. Med. 2013, 19 (12), 1597-1608. https://doi.org/10.1038/nm.3409.
Singh R.; Gupta P.; Sharma P. K.; Ades E. W.; Hollingshead S. K.; Singh S.; Lillard J. W. Prediction and characterization of helper T-cell epitopes from pneumococcal surface adhesin A. Immunology. 2014 April; 141(4):514-30. https://doi.org/10.1111/imm.12194.
Wang H, Yu F, Peng Y, Wang Q, Han X, Xu R, Zhou X, Wan C, Fan Z, Jiao P, Zhang Y, Zhang L, Zhou D, Xiao S. Synthesis and biological evaluation of ring A and/or C expansion and opening echinocystic acid derivatives for anti-HCV entry inhibitors. Eur J Med Chem. 2015 Sep. 18; 102:594-9. https://doi.org/10.1016/j.ejmech.2015.08.034.
Wang R. F. Identification of MHC class II-restricted tumor antigens recognized by CD4+ T cells. Methods. 2003 March; 29(3):227-35. https://doi.org/10.1016/s1046-2023 (02) 00345-6.
Wang Z, Zhou L, El-Boubbou K, Ye X S, Huang X. Multi-component one-pot synthesis of the tumor-associated carbohydrate antigen Globo-H based on preactivation of thioglycosyl donors. J Org Chem. 2007 Aug. 17; 72(17):6409-20. https://doi.org/10.1021/jo070585 g.
Wu Z, Guo X, Guo Z. Chemoenzymatic synthesis of glycosylphosphatidylinositol-anchored glycopeptides. Chem Commun (Camb). 2010 Aug. 21; 46(31):5773-4. https://doi.org/10.1039/c0cc00828a.
Yu W, Baskin J M. Photoaffinity labeling approaches to elucidate lipid-protein interactions. Curr Opin Chem Biol. 2022 August; 69:102173. https://doi.org/10.1016/j.cbpa.2022.102173 WO2009/126737, WO2015/184451, WO2017/079582, WO2017/106836, WO2018/191598, WO2018/200645 WO2018/200656 and WO2019/079160.

1. A compound of general formula (I) or a pharmaceutically acceptable salt thereof: 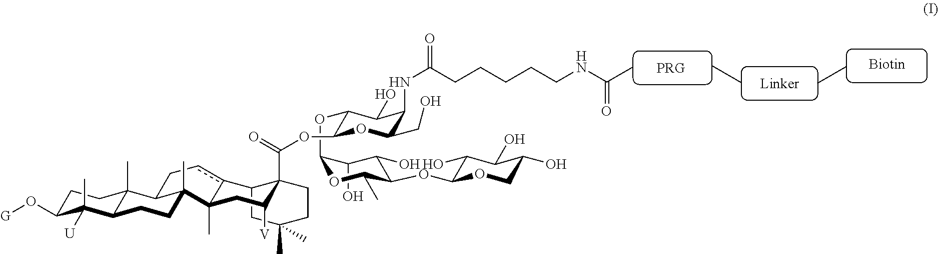
wherein G is a branched trisacchride of formula (VI) or a stereoisomer of formula (VI) 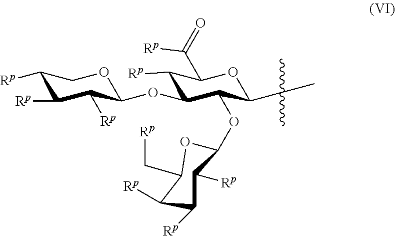
wherein each occurrence of RP is independently hydrogen or OR^{q};
wherein each occurrence of R^{q} is independently hydrogen or an optionally substituted group selected from 6-10-membered aryl, benzyl, C₁₋₆ aliphatic, or C₁₋₆ heteroaliphatic having 1-2 heteroatoms independently selected from the group consisting of nitrogen, oxygen, and sulfur; or two R^{q} are taken together to form a 5-7-membered heterocyclic ring having 1-2 heteroatoms independently selected from the group consisting of nitrogen, oxygen, and sulfur; [symbol]
is a single or double bond;
U is —CH₃, —CH₂—OH, —C═O or —CH═NOH;
V is H or OR^{x};
wherein R^{x} is independently hydrogen or an oxygen protecting group selected from the group consisting of alkyl ethers, benzyl ethers, silyl ethers, acetals, ketals, esters, carbamates, and carbonates;
PRG is a moiety comprising a photoreactive group selected from diazirine, benzophenone azido or a combination thereof;
Linker is a chemical spacer; and
Biotin is a biotin moiety.

2. The compound according to claim 1,
wherein G is

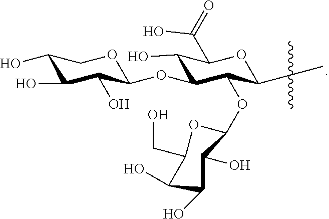

3. The compound according to claim 1, wherein U is —C═O.

4. The compound according to claim 1, wherein V is H or OH.

5. The compound according to claim 1, wherein PRG is a moiety comprising a photoreactive group selected from an aryldiazirine, a benzophenone, an arylazide or a combination thereof.

6. The compound according to claim 1 which is selected from:

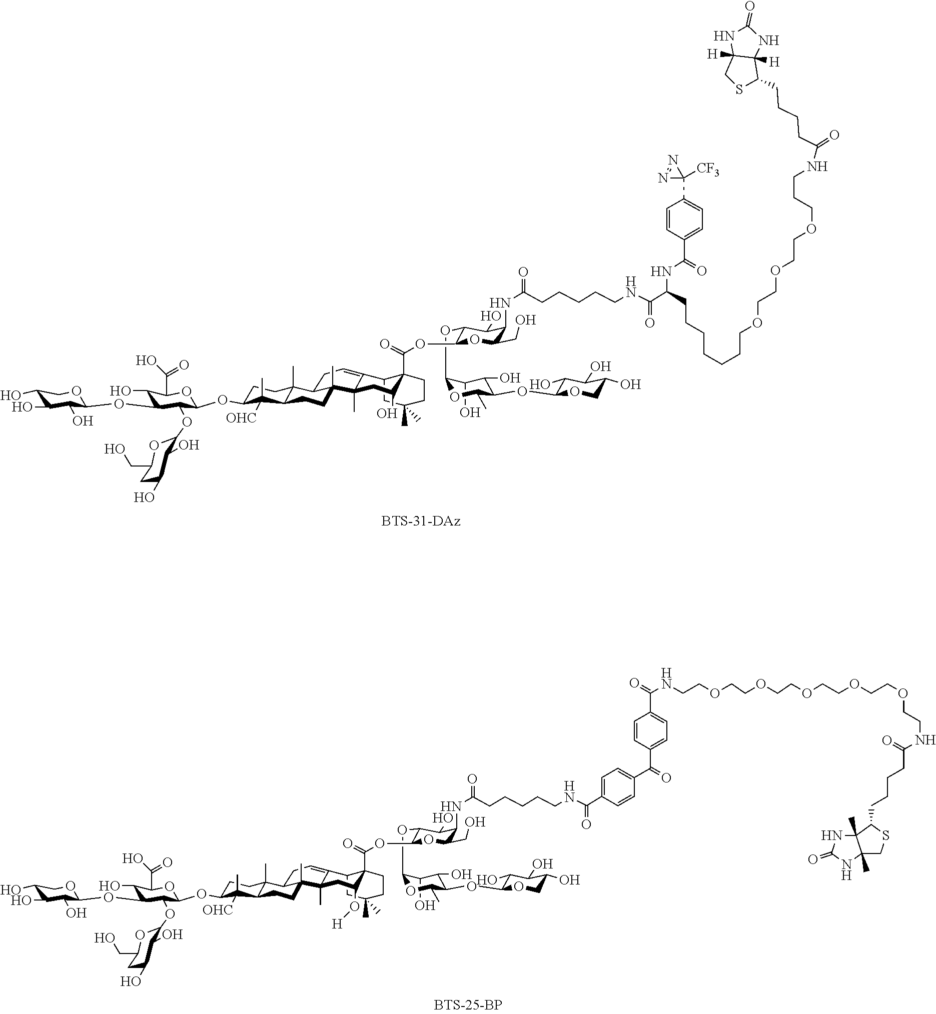

7. A method of synthesizing a compound of general formula (I) or a pharmaceutically acceptable salt thereof according to claim 1, said method comprising reacting: 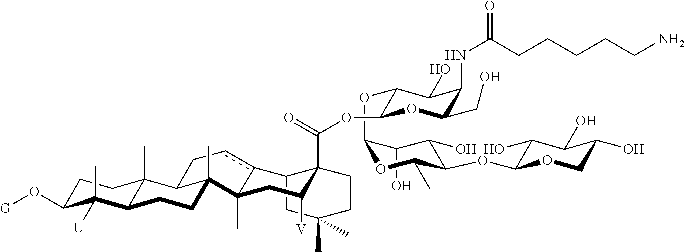
wherein G, [symbol] , U and V take the meanings as previously defined in formula (I);
with 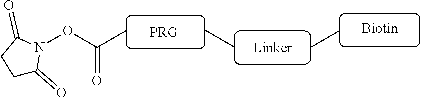
wherein PRG, linker and biotin take the meanings as previously defined in formula (I).

8. A pharmaceutical composition comprising a compound of general formula (I) or a pharmaceutically acceptable salt thereof according to claim 1, a pharmaceutically acceptable carrier and an antigen.

9. (canceled)

10. A compound of general formula (I) or a pharmaceutically acceptable salt thereof according to claim 1 for use in the treatment and/or prevention of cancer, an infectious disease or a neurodegenerative disease.

11. A compound of general formula (I) or a pharmaceutically acceptable salt thereof for use as chemical probe in photoaffinity labeling (PAL) experiments.

12. A pharmaceutical composition according to claim 8 for use in the treatment and/or prevention of cancer, an infectious disease or a neurodegenerative disease.
